(19) United States
(12) Patent Application Publication
(10) Pub. No.: US 2017/0246215 A1
(43) Pub. Date: Aug. 31, 2017

(54) REPOPULATION OF ORGANS AND TISSUES USING A YAP-ERT2 FUSION PROTEIN

(71) Applicant: Albert Einstein College of Medicine, Inc., Bronx, NY (US)

(72) Inventors: David A. Shafritz, Larchmont, NY (US); Mladen I. Yovchev, Pittsburgh, PA (US)

(73) Assignee: Albert Einstein College of Medicine, Inc., Bronx, NY (US)

(21) Appl. No.: 15/510,915

(22) PCT Filed: Sep. 11, 2015
(86) PCT No.: PCT/US2015/049768

§ 371 Date: Mar. 13, 2017

Related U.S. Application Data
(60) Provisional application No. 62/051,214, filed on Sep. 16, 2014.

(51) Int. Cl. A61K 35/407; A61K 38/17

(52) U.S. Cl. CPC A61K 35/407; A61K 38/1796

(57) ABSTRACT

Provided herein are methods for the repopulation of organs and tissues, such as the liver, using modified cells that express a transcription coactivation factor-ligand binding domain fusion protein, such as a YAP-ERT2 fusion protein. Also provided are compositions, including nucleic acid molecules that encode a YAP-ERT2 fusion protein, YAP-ERT2 fusion polypeptides, and cells containing nucleic acid molecules that encode a YAP-ERT2 fusion protein.

### CROSS-REFERENCE TO RELATED APPLICATION

This application claims the benefit of U.S. Provisional Application No. 62/051,214, filed Sep. 16, 2014, the contents of which are incorporated herein by reference in their entirety.

### BACKGROUND OF THE INVENTION

The only effective therapy currently available for end-stage liver disease is liver transplantation. The number of patients on the liver transplant list far exceeds the number of donor organs available (1). Although adult hepatocytes can engraft into the liver, they do not significantly repopulate the normal or regenerating normal liver (2). In contrast, fetal liver stem/progenitor cells (FLSPC), which have much higher proliferative activity than differentiated mature hepatocytes, can repopulate the normal liver and with a high number of transplanted FLSPC up to 20-25% of hepatic mass can be replaced (2, 3). However, use of FLSPC for liver cell therapy requires a high number of cells for repopulation, and the need for cells pooled from multiple donors will result in increased immunorejection. Further, there are ethical concerns regarding acquisition of fetal liver cells for human use.

Transplanted fetal liver stem/progenitor cells FLSPC repopulate the liver by proliferating faster than host hepatocytes and induce apoptosis in the latter (3). This process is referred to as “cell competition,” a process that was originally described in Drosophila during wing development (4,5). Since transplanted mature hepatocytes exhibit little, if any, difference in proliferative activity or survival advantage compared to host hepatocytes, they do not significantly repopulate the liver, except under most adverse circumstances in which there is massive and continuous liver injury in the host (e.g. in uPA transgenic or FAH null mice (6,7)) or the ability of host hepatocytes to proliferate is markedly impaired (e.g., DNA damage induced by DNA crosslinking agents, such as retrorsine, monocrotaline or x-irradiation (8-10). Treatment with such toxic agents is undesirable for use in clinical protocols to repopulate the liver by hepatocyte transplantation.

### SUMMARY OF THE INVENTION

Provided herein, in certain embodiments, are methods for repopulation of organs and tissues with modified cells obtained from normal deceased or living donors that are modified to increase their rate of cellular proliferation. In some embodiments, the modified cells express a Yes-associated protein-estrogen receptor 2 (YAP-ERT2) fusion protein.

Described herein, in certain embodiments are methods for repopulating the liver in a patient having a liver disease or condition, comprising: (a) transplanting a plurality of modified normal liver cells into the liver of the patient having a liver disease or condition, wherein the modified cells comprise a nucleic acid molecule encoding a Yes-associated protein-estrogen receptor 2 (YAP-ERT2) fusion protein; and (b) administering an estrogen receptor antagonist to the patient, wherein the estrogen receptor antagonist increases the proliferative activity of the modified cells, thereby repopulating the patient's liver with the modified cells. In some embodiments, the normal liver cells are obtained from a deceased or living donor. In some embodiments, administration of the estrogen receptor antagonist induces nuclear translocation of the YAP-ERT2 fusion protein where it functions as a transcriptional coactivator of Yap target genes. In some embodiments, YAP-ERT2 is retained in the cytoplasm of the cells in the absence of the estrogen receptor antagonist. In some embodiments, administration of the modified cells comprises transplantation of the cells into the liver of the patient. In some embodiments, the modified cells repopulate the liver cell population in the patient by about 1% or greater at about one month following administration of the modified cells. In some embodiments, the modified cells repopulate the liver cell population in the patient by about 3-5% or greater at about three months following administration of the modified cells. In some embodiments, the modified cells repopulate the liver cell population in the patient by about 8-12% or greater at about six months following administration of the modified cells. In some embodiments, modified cells are administered by injection into the spleen or portal vein. In some embodiments, about 1-10×10⁹ modified cells modified cells are administered to the patient. In some embodiments, the estrogen receptor antagonist is administered at a dosage of about 10 mg/day to about 100 mg/day. In some embodiments, the estrogen receptor antagonist is administered at a dosage of about 20 mg/day to about 40 mg/day. In some embodiments, the estrogen receptor antagonist is tamoxifen. In some embodiments, the 4-hydroxytamoxifen metabolite of tamoxifen binds to the ERT2 portion of the YAP-ERT2 fusion protein. In some embodiments, tamoxifen is administered at a dosage of about 10 mg/day to about 100 mg/day. In some embodiments, tamoxifen is administered at a dosage of about 20 mg/day to about 40 mg/day. In some embodiments, the estrogen receptor antagonist is administered for 1, 2, 3, 4, 5, 6, 7, 8, 9, 10, 11, 12 months or longer. In some embodiments, the YAP-ERT2 fusion protein exhibits low or no binding affinity for 17β-estradiol. In some embodiments, the estrogen receptor antagonist is administered once a day or twice a day. In some embodiments, the estrogen receptor antagonist is administered orally. In some embodiments, the estrogen receptor antagonist is administered simultaneously with the modified cells. In some embodiments, the estrogen receptor antagonist is administered about 6, 12, 18, 24, 36, or 48 hours following administration of the modified cells. In some embodiments, the nucleic acid molecule encoding the YAP-ERT2 fusion protein comprises a nucleotide sequence set forth in SEQ ID NO: 1. In some embodiments, the YAP-ERT2 fusion protein comprises a polypeptide having the amino acid sequence set forth in SEQ ID NO: 2. In some embodiments, the nucleic acid molecule encoding the YAP-ERT2 fusion protein is operably linked to a promoter. In some embodiments, the promoter is a ubiquitous promoter or a cell-specific promoter. In some embodiments, the promoter is a liver-specific promoter. In some embodiments, the promoter is a transthyretin (TTR) promoter. In some embodiments, the cells are primary hepatocytes or a hepatic cell line. In some embodiments, the primary hepatocytes are derived from a deceased or living donor. In some embodiments, the primary hepatocytes are derived from the patient. In some embodiments, the cells are stem cells. In some embodiments, the stem cells are embryonic stem (ES) cells or induced pluripotent stem (iPS) cells. In some embodiments, the cells are generated by viral transduction of the cells. In some embodiments, the virus is selected from among a lentivirus, a retrovirus, adenovirus, adeno-associated virus, or Sendai virus. In some embodiments, the cells are generated using zinc finger nuclease (ZFN), a transcription activator-like effector nuclease (TALEN), or clustered regulatory interspaced short palindromic repeat (CRISPR)/Cas-based RNA-guided DNA endonuclease technology, a recombinant Epstein Barr Nuclear Antigen plasmid, or a self-replicating RNA molecule. In some embodiments, the liver disease or condition is a genetic based disease. In some embodiments, the liver disease or condition is selected from genetic-based liver diseases in which there is no underlying or ongoing liver injury, including, but not limited to UDP-glucuronosyl transferase deficiency (Crigler-Najjar Syndrome, Type 1), ornithine transcarbamylase deficiency, Familial Hypercholesterolemia, phenylketonuria, Hemophilia B, Factor VII deficiency, primary hyperoxaluria, maple syrup urine disease and Apolipoprotein E deficiency. In some embodiments, the modified cells obtained from normal or deceased liver are transplanted into the liver of a patient having a genetic-based liver disease or condition in which there is underlying liver injury and ongoing liver damage, selected from but not limited to Wilson's Disease, α1-antitrypsin deficiency Hereditary, Hemochromatosis, Progressive Familial Intrahepatic Cholestasis (Types I, II and III), and Bile Salt Export Protein deficiency. In some embodiments, the liver disease or condition is a non-genetic based chronic liver disease. In some embodiments, the liver disease or condition is selected from among patients with non-alcoholic fatty liver disease and chronic hepatitis C virus infection. In some embodiments, the patient exhibits hepatic fibrosis or cirrhosis. In some embodiments, the patient is a human patient. In some embodiments, the patient has elevated serum bilirubin levels resulting from UDP-glucuronosyl transferase deficiency (Crigler-Najjar Syndrome, Type 1) prior to administration of the modified cells. In some embodiments after administration of modified cells, the level of serum bilirubin in the patient with Crigler-Najjar Syndrome, Type 1, is decreased compared to pre-treatment levels by 50% or greater at 90 days following administration of the modified cells and returns to normal by 180 days. In some embodiments, the methods further comprise administration of an immunosuppressant. In some embodiments, the methods further comprise administration of an additional therapeutic agent. In some embodiments, the therapeutic agent is an anti-fibrotic agent. In some embodiments, the anti-fibrotic agent includes, but is not limited to, Sorafemib, largazole, galectin inhibitors, FG-3019 (an anti-CTGF antibody) Pirfenidone, a TGF-I3 inhibitor, endostatin peptide, and Polarezin. In some embodiments, the methods further comprise modifying the frequency or dosage of the estrogen receptor antagonist administered to the patient over the course of treatment. In some embodiments, modifying comprises increasing or decreasing the frequency or dosage of the estrogen receptor antagonist administered to the patient. In some embodiments, the methods further comprise modifying the frequency or dosage of the estrogen receptor antagonist based on the serum bilirubin levels in a patient having Crigler-Najjar Syndrome, Type 1. In some embodiments, the modified cells further express a therapeutic gene. In some embodiments, the therapeutic gene is deficient in the patient. In some embodiments, the modified cells are generated from primary hepatocytes obtained from the patient, wherein the patient has a genetic-based liver disease or condition but no underlying or ongoing liver injury. In some embodiments, the genetic-based liver disease or condition in which there is no underlying liver injury or ongoing liver damage is selected from among, but not limited to UDP-glucuronosyl transferase deficiency (Crigler-Najjar Syndrome, Type 1), ornithine transcarbamylase deficiency, Familial Hypercholesterolemia, phenylketonuria, Hemophilia B, Factor VII deficiency, primary hyperoxaluria, maple syrup urine disease and Apolipoprotein E deficiency. In some embodiments, the modified cells obtained from a normal or deceased donor are transplanted into the liver of a patient having a genetic-based liver disease or condition in which there is underlying liver injury and ongoing liver damage selected from, but not limited to, Wilson's Disease, al antitrypsin deficiency, Hereditary Hemochromatosis, Progressive Familial Intrahepatic Cholestasis, Types I, II and III, and Bile Salt Export Protein deficiency.

Described herein, in certain embodiments, are methods for repopulating the liver in a patient having a genetic-based liver disease or condition, comprising: (a) administering a plurality of modified cells obtained from the patient, wherein the modified cells comprise: (i) a nucleic acid molecule encoding a Yes-associated protein-estrogen receptor 2 (YAP-ERT2) fusion protein and (ii) a nucleic acid molecule encoding a protein that is deficient or defective in the patient; (b) transplanting the modified cells back into the liver of the patient (autologous cell transplantation); and (c) administering an estrogen receptor antagonist, wherein the estrogen receptor antagonist increases the proliferative activity of the modified cells, thereby repopulating the patient's liver with the modified cells. In some embodiments, administration of the estrogen receptor antagonist induces nuclear translocation of the YAP-ERT2 fusion protein where it functions as a transcriptional coactivator of Yap target genes. In some embodiments, the YAP-ERT2 is retained in the cytoplasm of the cells in the absence of the estrogen receptor antagonist. In some embodiments, administration of the modified cells comprises transplantation of the cells into the liver of the patient. In some embodiments, the modified cells repopulate the liver cell population in the patient by about 1% or greater at about one month following administration of the modified cells. In some embodiments, the modified cells repopulate the liver cell population in the patient by about 3-5% or greater at about three months following administration of the modified cells. In some embodiments, the modified cells repopulate the liver cell population in the patient by about 8-12% or greater at about six months following administration of the modified cells. In some embodiments, the modified cells are administered by injection into the spleen or portal vein. In some embodiments, about 1-1×10⁹ modified cells are administered to the patient. In some embodiments, the estrogen receptor antagonist is administered at a dosage of about 10 mg/day to about 100 mg/day. In some embodiments, the estrogen receptor antagonist is administered at a dosage of about 20 mg/day to about 40 mg/day. In some embodiments, the estrogen receptor antagonist is tamoxifen. In some embodiments, the 4-hydroxytamoxifen metabolite of tamoxifen binds to the ERT2 portion of the YAP-ERT2 fusion protein. In some embodiments, tamoxifen is administered at a dosage of about 10 mg/day to about 100 mg/day. In some embodiments, tamoxifen is administered at a dosage of about 20 mg/day to about 40 mg/day. In some embodiments, the estrogen receptor antagonist is administered for 1, 2, 3, 4, 5, 6, 7 , 8, 9, 10, 11, 12 months or longer. In some embodiments, the YAP-ERT2 fusion protein exhibits low or no binding affinity for 17β-estradiol. In some embodiments, the estrogen receptor antagonist is administered once a day or twice a day. In some embodiments, the estrogen receptor antagonist is administered orally. In some embodiments, the estrogen receptor antagonist is administered simultaneously with the modified cells. In some embodiments, the estrogen receptor antagonist is administered about 6, 12, 18, 24, 36, 48, 60 or 72 hours following administration of the modified cells. In some embodiments, the nucleic acid molecule encoding the YAP-ERT2 fusion protein comprises a nucleotide sequence set forth in SEQ ID NO: 1. In some embodiments, the YAP-ERT2 fusion protein comprises a polypeptide having the amino acid sequence set forth in SEQ ID NO: 2. In some embodiments, the nucleic acid molecule encoding the YAP-ERT2 fusion protein is operably linked to a promoter. In some embodiments, the promoter is a ubiquitous promoter or a cell-specific promoter. In some embodiments, the promoter is a liver-specific promoter. In some embodiments, the promoter is a transthyretin (TTR) promoter. In some embodiments, the cells are primary hepatocytes or a hepatic cell line. In some embodiments, the methods further comprise the primary hepatocytes are derived from the patient. In some embodiments, the cells are stem cells. In some embodiments, the stem cells are embryonic stem (ES) cells or induced pluripotent stem (iPS) cells. In some embodiments, the cells are generated by viral transduction of the cells. In some embodiments, the virus is selected from among a lentivirus, a retrovirus, adenovirus, adeno-associated virus, Sendai virus. In some embodiments, the cells are generated using zinc finger nuclease (ZFN), a transcription activator-like effector nuclease (TALEN), or clustered regulatory interspaced short palindromic repeat (CRISPR)/Cas-based RNA-guided DNA endonuclease technology, a recombinant Epstein Barr Nuclear Antigen plasmid, or a self-replicating RNA molecule. In some embodiments, the genetic based liver disease or condition is selected from among, but not limited to UDP-glucuronosyl transferase deficiency (Crigler-Najjar Syndrome, Type 1), ornithine transcarbamylase deficiency, Familial Hypercholesterolemia, phenylketonuria, Hemophilia B, Factor VII deficiency, primary hyperoxaluria, maple syrup urine disease, Apolipoprotein E deficiency, Wilson's Disease, α1-antitrypsin deficiency, Hereditary Hemochromatosis, Progressive Familial Intrahepatic Cholestasis (Types I, II and III), and Bile Salt Export Protein deficiency. In some embodiments, the patient exhibits hepatic fibrosis or cirrhosis. In some embodiments, the patient has a normal or near-normal liver. In some embodiments, the patient is a human patient. In some embodiments, the patient has elevated serum bilirubin levels resulting from UDP-glucuronosyl transferase deficiency (Crigler-Najjar Syndrome, Type 1) prior to administration of the modified cells. In some embodiments, the level of serum bilirubin in the patient with Crigler-Najjar Syndrome, Type 1, is decreased compared to pre-treatment levels by 50% or greater at 90 days following administration of the modified cells. In some embodiments, the methods further comprise administration of an immunosuppressant. In some embodiments, the methods further comprise administration of an additional therapeutic agent. In some embodiments, the therapeutic agent is an anti-fibrotic agent. In some embodiments, the anti-fibrotic agent is selected from among, but not limited to Sorafemib, largazole, galectin inhibitors, FG-3019 (an anti-CTGF antibody) Pirfenidone, a TGF-β inhibitor, endostatin peptide, and Polarezin. In some embodiments, the methods further comprise modifying the frequency or dosage of the estrogen receptor antagonist administered to the patient over the course of treatment. In some embodiments, the methods further comprise increasing or decreasing the frequency or dosage of the estrogen receptor antagonist administered to the patient. In some embodiments, the methods further comprise modifying the frequency or dosage of the estrogen receptor antagonist based on the serum bilirubin levels in a patient having Crigler-Najjar Syndrome, Type 1.

Described herein, in certain embodiments, are methods for treating diabetes in a patient, comprising: (a) administering to the patient a plurality of modified ES or iPS cells differentiated along the pancreatic islet β cell lineage pathway, wherein the modified pancreatic islet β cells comprise a nucleic acid molecule encoding a Yes-associated protein-estrogen receptor 2 (YAP-ERT2) fusion protein; and (b) administering an estrogen receptor antagonist that activates Yap ERT2 fusion protein function and induces cell proliferation, thereby treating the diabetes. In some embodiments, the nucleic acid encoding the YAP-ERT2 fusion protein is operably linked to an islet β cell-specific promoter. In some embodiments, the promoter is an insulin promoter.

Described herein, in certain embodiments, are isolated nucleic acid molecules encoding the fusion protein comprising a Yes-associated protein (YAP) and an estrogen receptor 2 (ERT2). In some embodiments, the isolated nucleic acid has a nucleotide sequence as set forth in SEQ ID NO: 1. In some embodiments, the nucleic acid molecule is operably linked to a promoter. In some embodiments, the promoter is a ubiquitous promoter or a cell-specific promoter. In some embodiments, the nucleic acid molecule is operably linked to a pancreatic islet β cell-specific promoter. In some embodiments, the promoter is (TTR) promoter or an insulin promoter.

Described herein, in certain embodiments, are methods for repopulating the cells of a normal tissue or organ comprising: introducing a plurality of modified cells into a tissue or organ sufficient to effect a 3% or greater repopulation of the cells in the tissue or organ under non-selective conditions within about 3-6 months, wherein the modified cells are modified to increase their proliferative potential compared to unmodified cells in the presence of a ligand; and optionally, administering the ligand to the modified cells. In some embodiments, administration of the ligand promotes the nuclear translocation of an exogenous protein expressed by the modified cells. In some embodiments, the exogenous protein is a fusion protein comprising a nuclear transcription coactivation factor that promotes cell proliferation and a ligand binding domain. In some embodiments, the ligand binding domain keeps the fusion protein in the cytoplasm to prevent its function in the absence of the ligand. In some embodiments, administration of the ligand promotes the nuclear translocation of the fusion protein to the nucleus, whereby its function as a transcriptional coactivation factor is induced and cells containing the fusion-protein are activated to proliferate and repopulate the host tissue. In some embodiments, the transcription coactivation factor is Yes-associated protein (YAP). In some embodiments, the ligand binding domain is an estrogen receptor 2 (ERT2) ligand binding domain. In some embodiments, the ligand is a tamoxifen metabolite.

Described herein, in certain embodiments, are methods for repopulating the liver in a patient having a liver disease or condition, comprising: (a) isolating primary hepatocytes from a patient having a liver disease or condition; (b) modifying the primary hepatocytes by introducing a nucleic acid molecule encoding a Yes-associated protein-estrogen receptor 2 (YAP-ERT2) fusion protein and a second nucleic acid molecule containing a gene that is deficient in the liver of the patient; (c) administering the modified cells to the patient; and (d) administering an estrogen receptor antagonist, wherein the estrogen receptor antagonist increases the proliferative activity of the modified cells, thereby repopulating the liver with the modified cells.

Described herein, in certain embodiments, are methods for repopulating the cells of a tissue or organ in a patient, comprising: (a) administering a plurality of modified cells to the tissue or organ, wherein the modified cells comprise a nucleic acid molecule encoding a fusion protein comprising a nuclear transcription coactivation factor that promotes cell proliferation and a steroid binding domain; and (b) administering a steroid binding domain antagonist, wherein the steroid binding domain antagonist increases the proliferative activity of the modified cells by promoting the nuclear translocation of the transcription coactivation factor, thereby repopulating the tissue or organ with the modified cells. In some embodiments, the nuclear transcription coactivation factor is Yes-associated protein (YAP). In some embodiments, the steroid binding domain is an estrogen receptor ligand binding domain. In some embodiments, the steroid binding domain antagonist is tamoxifen.

Described herein, in certain embodiments, are isolated vectors comprising any nucleic acid molecule encoding a fusion protein comprising a Yes-associated protein (YAP) and an estrogen receptor 2 (ERT2) provided herein. In some embodiments, the vector is a virus vector, a plasmid vector, or a self-replicating RNA vector. In some embodiments, the virus vector is selected from among a lentivirus, a retrovirus, adenovirus, adeno-associated virus, or Sendai virus.

Described herein, in certain embodiments, are isolated cells comprising any vector or nucleic acid molecule encoding a fusion protein comprising a Yes-associated protein (YAP) and an estrogen receptor 2 (ERT2) provided herein. In some embodiments, the cell is a primary hepatocyte, a hepatic cell line, a pancreatic islet 0 cell or a pancreatic islet β cell line. In some embodiments, the cell is a stem cell. In some embodiments, the stem cell is an embryonic stem (ES) cell or an induced pluripotent stem (iPS) cell.

Described herein, in certain embodiments, are isolated fusion proteins comprising a Yes-associated protein (YAP) and an estrogen receptor 2 (ERT2). In some embodiments, the isolated fusion protein has an amino acid sequence set forth in SEQ ID NO: 2.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 illustrates essential genes and steps in mammalian Hippo signaling.

FIG. 2 illustrates expression of core Hippo signaling genes in FLSPC vs. adult liver. A) Heat maps from microarray analysis indicating the relative ratio for expression of Mst½, Lats½, Yap and survivin (Birc5) in purified mouse FLSPC vs. adult mouse liver. Ms½, decreased 3.0 fold; Lats^{1/2}, unchanged; Yap, increased 2.0 fold; survivin, increased 20 fold. For Yap, 3 different ESTs were present on the microarray chips. Lanes 1, 2 and 3 represent data from sample pairs from 3 separate experiments. B) RT-PCR for mRNA expression of anti-apoptotic genes in purified rat FLSPC (Fl) vs. adult hepatocytes (Hc), using GAPDH as loading control. Anti-apoptotic genes Bc1-2 and mcl are not expressed in rats and were not included in this analysis.

FIG. 3 illustrates lentivirus transgene constructs used in the examples.

FIG. 4 illustrates transduction of HeLa cells with lentivirus vectors. Transduced HeLa cells were assayed at day 4 in culture for expression of the indicated transgene. A) lenti EF1-GFP; expression in both cytoplasm & nucleus B) lenti EF1-H2B GFP-hYap; Yap expression in nucleus C) lenti EF1-YAP-ERT2 (−) tamoxifen; Yap expression almost exclusively in cytoplasm D) lenti EF1-hYAP-ERT2 (+) tamoxifen; Yap expression predominantly in nucleus. This experiment validates that the subcellular location of hYapERT2 in virally transduced cells is regulated by tamoxifen.

FIG. 5 illustrates expression of Yap target genes in HeLa cells and adult rat hepatocytes transduced with lenti EF1-hYAP-ERT2. Fold changes in mRNAs quantified by q RT PCR in absence (set as 1) vs. presence of 0.1 μM 4-OH tamoxifen for 4 days. This experiment validates that the function of hYapERT2 in inducing downstream target genes is regulated by tamoxifen.

FIG. 6 illustrates liver repopulation by lenti TTR-hYapERT2 transduced hepatocytes transplanted into normal adult liver. A) DPPIV⁺ clusters at 3 months in a DPPIV⁻ rat fed tamoxifen containing chow, 500 mg/kg (+Tam). Repopulation=3.2% by quantitative image analysis of whole section. B) Rare scattered DPPIV⁻ single cells, doublets and groups of 3-4 cells at 3 months in rat transplanted with the same lenti TTR-hYapERT2 transduced hepatocyte preparation used in A, but fed a normal chow diet (−Tam) Repopulation <0.1% by quantitative image analysis of whole section. C) DPPIV⁺ clusters at 6 months in a DPPIV⁻ rat fed tamoxifen containing chow, 500 mg/kg (+Tam). Repopulation=15.6% by quantitative image analysis of whole section. D) Rare scattered DPPIV⁺ single cells, doublets and groups of 3-4 cells at 6 months in DPPIV⁻ rat transplanted with the same lenti TTR-hYapERT2 transduced hepatocyte preparation used in A, but fed a normal chow diet (−Tam) Repopulation=0.1% by quantitative image analysis of whole section.

FIG. 7 illustrates incorporation of transplanted, lenti TTR-hYapERT2 transduced hepatocytes into the liver parenchymal plates. Animals sacrificed at 6 months after hepatocyte transplantation and maintained on tamoxifen diet and A) & C) H & E staining of a representative section of repopulated rat liver at orig. mag. 4× and 20×, respectively. B) and D) serial section of the same liver region stained for DPPIV at orig. mag. 4× and 20×, respectively. On both H&E and DPPIV staining, liver tissue post lenti TTR-YapERT2 transduced hepatocyte transplantation was totally normal and the transplanted cells and their progeny were fully incorporated into a totally normal liver structure.

FIG. 8 illustrates double label fluorescence immunohistochemistry for DPPIV and selected marker genes in repopulating clusters at 6 months after lenti TTR-hYap-ERT2 transduced hepatocyte transplantation. The specific protein paired with DPPIV and the fluorescent color generated by the secondary antibody for each protein are indicated in panels A-L. Nuclei are stained with Dapi. Orig. mag.=60×.

FIG. 9 illustrates repopulation of the normal adult liver by rat hepatocytes transduced with lenti TRR-hYap-ERT2 vs. lenti TTR-GFP vs. no virus. In these experiments, 5×10⁶ lenti TTR-hYap-ERT2 transduced, lenti TTR-GTP transduced or mock transduced (no virus) WT 344 rat hepatocytes (DPPIV⁺) were transplanted into DPPIV⁻ F344 rats according to our standard protocol. Cell transplantation recipients were maintained on a 500 mg/kg tamoxifen containing chow diet for 6 months (+tam). For recipients transplanted with lenti TTR-YapERT2 transduced hepatocytes, a separate group of rats was maintained on a normal rodent chow diet (−tam). The animals were then sacrificed, liver tissue (two sections each from at least 3 lobes of each liver) was stained for DPPIV⁺ expression by enzyme histochemistry and the % repopulation by DPPIV⁺ hepatocytes quantified by analysis of digital images using a Zeiss Axio Observer Z1 microscope and Image J software. Three animals were included in each group. Data are shown as mean±SEM. Statistical significance was determined by a 2-tailed Student's t-test.

FIG. 10 illustrates repopulation of the liver at 1 year following transplantation of lenti TTR-hYapERT2 transduced hepatocytes. WT DPPIV⁺ rat hepatocytes transduced with lenti TTR-hYapERT2 at 500 VP/cell for 4 hours at room temperature were transplanted into the spleen of a DPPIV⁻ rat recipient. Liver repopulation, determined by DPPIV expression by enzyme histochemistry, using quantitative imaging was: A) 17.5% repopulation in lobe 1 and B) 46.8% repopulation in lobe 3.

FIG. 11 illustrates a schematic diagram of the expected reduction in serum bilirubin in Gunn rats transplanted with lenti TTR-hYap-ERT2 transduced WT rat hepatocytes. Gunn rats will be transplanted with 5×10⁶ WT hepatocytes transduced with lenti TTR-hYap-ERT2 according to our standard procedure. In Protocol 1, the rats will be maintained on tamoxifen chow for 6 months (+T), after which normal rat chow will be given for an additional 6 months (−T). Serum bilirubin will be determined weekly or semi-weekly with an initial value of ˜7 mg/dl. In the schematic diagram, this is indicated as 100%. Serum bilirubin is expected to decrease progressively over time, as replacement of host hepatocytes by lenti TTR-hYap-ERT2 hepatocytes occurs, while the rats are on the tamoxifen diet. Based on results with the DPPIV⁺ model, we expect a 75-80% decrease in serum bilirubin in 6 months on tamoxifen feed. When tamoxifen is discontinued, the serum bilirubin level should remain constant but may decrease further if repopulation continues ( [symbol] ) or increase if repopulating cells are lost ( [symbol] ). In Protocol 2, we will treat lenti TTR-hYap-ERT2 WT hepatocyte transplanted Gunn rats with tamoxifen feed for 3 months after which we expect a 50% reduction in serum bilirubin. The animals will then be maintained on a normal chow diet for 6 months during which time we expect the serum bilirubin to remain stable. However, as noted in Protocol 1, serum bilirubin may decrease further if the % of liver repopulation by transplanted lenti TTR-hYap-ERT2 hepatocytes increases ( [symbol] ), or it could increase if lenti TTR-hYap-ERT2 transduced WT hepatocytes are lost ( [symbol] ). After 6 months on normal chow, the rats will be re-fed with the tamoxifen containing diet. We expect a resumption in the decrease in serum bilirubin, resulting from increased repopulation by lenti TTR-hYap-ERT2 WT hepatocytes. Such a result will demonstrate that we can control and modulate the level of liver repopulation to obtain effective therapy in a genetic-based liver disease and re-treat patients as necessary over time. In these experiments, animals will be sacrificed at various time points to determine the % liver repopulation by immunohistochemistry for UGT-1A1 and biochemical determination of UGT-1A1 enzyme activity in liver tissue homogenates.

FIG. 12 illustrates repopulation of the Gunn rat liver by Gunn rat hepatocytes transduced with both lenti TTR- UGT1A1- and lenti TTR-Yap ERT2. 5×10⁶ primary hepatocytes, isolated from Gunn rats and transduced with 500 VP/cell of both lenti TTR-UGT 1A1 and lenti TTR-Yap ERT2, will be transplanted into syngeneic Gunn rats according to our standard protocol. In Protocol 1 (−) T, the UGT 1A1 transduced cells will begin to lower the serum bilirubin as soon as UGT-1A1 protein is expressed (within 1-2 weeks). As reported previously (47), serum bilirubin will fall by ˜30% within the first month, but will not become lower, because in the absence of tamoxifen, the transplanted cells will not proliferate ( [symbol] ). However, in Protocol 2, (+) T/(−) T, in the presence of tamoxifen, transplanted cells will proliferate based on Yap ERT2 expression/function, and as repopulation increases over the next 6 mo., serum bilirubin will fall to normal levels ( [symbol] ). When the serum bilirubin becomes normal, tamoxifen administration will be discontinued and serum bilirubin will remain normal ( [symbol] ). Rats will be followed for up to 2 years with continuous monitoring of serum bilirubin. If the serum bilirubin rises in Protocol 2 after tamoxifen is withdrawn, tamoxifen will be restarted with the expectation that serum bilirubin will promptly return to normal.

FIG. 13 illustrates repopulation of the fibrotic/cirrhotic liver by transplanted fetal liver stem/progenitor cells (FLSPC) vs. adult hepatocytes. FLSPC or adult hepatocytes from DPPIV⁻ F344 rats were transplanted into DPPIV⁻ F344 rats in which dense fibrosis/cirrhosis was induced by 10-12 weeks of thioacetamide (TAA) administration. Animals transplanted with FLSPC or adult hepatocytes were maintained for 2 months, while continuing TAA treatment, were then sacrificed and liver tissue sections stained for DPPIV by enzyme histochemistry. The level of repopulation was 35-40% with FLSPC and 8-10% with adult hepatocytes. Data taken from ref 35. With transplanted lenti TTR-hYap-ERT2 transduced hepatocytes, we expect repopulation to increase to a level comparable to that obtained with FLSPC, which would be sufficient for an effective therapeutic response in terms of liver regeneration and function.

FIG. 14 illustrates the DNA sequence (SEQ ID NO: 1) and amino acid polypeptide sequence (SEQ ID NO: 2) of hYap ERT2 fusion protein. The TTR promoter is shown in red lower case letters above the hYapERT2 sequences which are shown in black upper case letters. In each paired row of letters, the upper letters are the DNA sequence (SEQ ID NO: 1) and the lower letters are the amino acid polypeptide sequence (SEQ ID NO: 2). The entire nucleotide sequence of the TTR promoter with the hYap ERT2 sequence is represented in the sequence listing as SEQ ID NO: 3.

### DETAILED DESCRIPTION OF THE INVENTION

Described herein is a method for liver repopulation based on increasing the proliferative potential of transplantable cells, including mature hepatocytes, by introducing a gene that induces the cell cycle and activates cellular proliferation. These modified cells are effective in liver repopulation under much less severe conditions than used previously (6-10) or even in a normal or near normal liver, and thus do not require the use of toxic agents to inhibit the host cells. This is an important issue because several major genetic-based disorders of the liver that have severe clinical consequences do not include any underlying liver injury or damage and, therefore, cannot be treated successfully by cell transplantation using normal hepatocytes (e.g., Crigler-Najjar Syndrome, Type1, which causes hyperbilirubinemia, brain damage and mental retardation (11), ornithine transcarboxylase deficiency, which causes ammonia toxicity, coma and death (12), LDL-receptor deficiency (Familial Hypercholesterolemia), which causes heart attacks and strokes in children and adolescents (13), phenylketonuria, which causes brain damage and mental retardation, hemophilia B (Factor IX deficiency), which causes spontaneous internal bleeding leading to joint damage, intracranial hemorrhage and death (14), and Primary hyperoxaluria, which causes severe renal disease and kidney failure (15), among others). However, these diseases can be treated according to the methods provided herein by transplanting modified normal hepatocytes, which carry a gene that when expressed, increases the proliferative potential of the cells transplanted into the liver. Accordingly, the methods provided herein achieve effective liver repopulation by cell transplantation in genetic disorders of the liver that would otherwise require a liver transplant, as well as to restore liver function in patients with chronic liver disease before development of end stage hepatic fibrosis/cirrhosis.

Within one or two months after birth, the liver structure is fully formed and subsequently the organ grows only in proportion to total body mass (16). In the adolescent and adult liver, the hepatocyte, which carries out the major metabolic functions of the liver, is in a non-growth, quiescent state and divides only 2-3×/yr as part of normal tissue turnover. However, after acute or during chronic liver injury or loss of liver mass by trauma or surgery, hepatocytes enter a growth state, proliferate rapidly and restore liver mass to normal within one week, after which the hepatocytes return to a quiescent state. Hepatocytes isolated from the normal liver can be maintained briefly in culture, but they do not grow. Such hepatocytes can be transplanted into a secondary host, after which they engraft and become incorporated into the liver structure (17). However, if the transplanted hepatocytes and host liver are normal, both cell types will respond equally to a liver regenerative stimulus and there will be no significant repopulation by transplanted hepatocytes.

In animal model systems in which the host liver has massive and continuous liver damage through introduction of a genetic defect, such as in uPA transgenic of FAH null mice (6,7), or host hepatocytes are mitoinhibited by chemical damage to the cellular DNA using cross-linking agents, such as retrorsine (8), monocrotaline (9) or x-irradiation (10), transplanted normal hepatocytes will have a selective advantage over host hepatocytes and will replace nearly all of the host liver within 6-8 weeks. However, treatment with such agents is undesirable for use in clinical protocols to repopulate the liver by hepatocyte transplantation.

One method to repopulate the normal liver is to use cells that have a proliferative advantage over host hepatocytes. In a rat model system, fetal liver stem/progenitor cells, which have a substantially higher proliferative capacity than adult hepatocytes, effectively repopulate the normal liver (2, 3). After repopulation, the liver structure is normal and there is no evidence for carcinogenesis produced by transplanted fetal liver cells for up to two years after cell transplantation. However, use of FLSPC for liver cell therapy has significant disadvantages in that it requires a high number of cells for repopulation, requires cells pooled from multiple donors which increases the risk of immunorejection and raises ethical concerns.

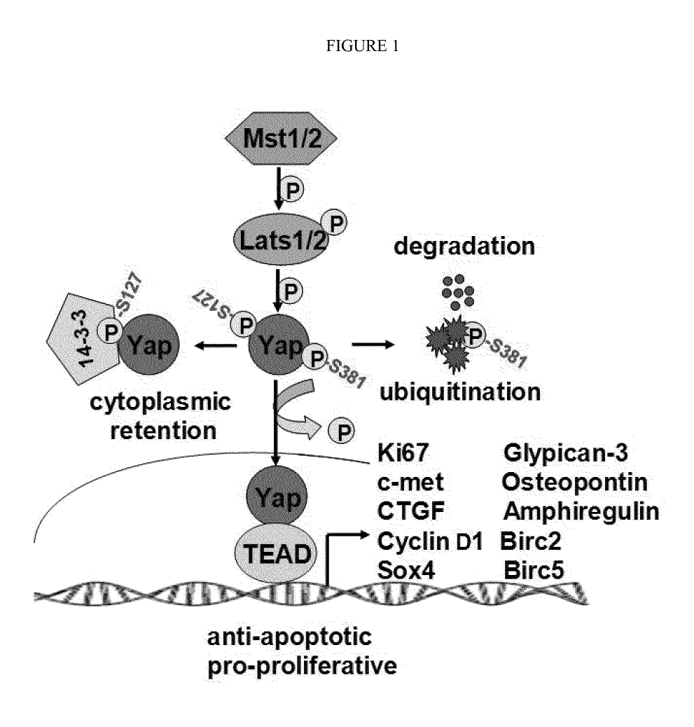

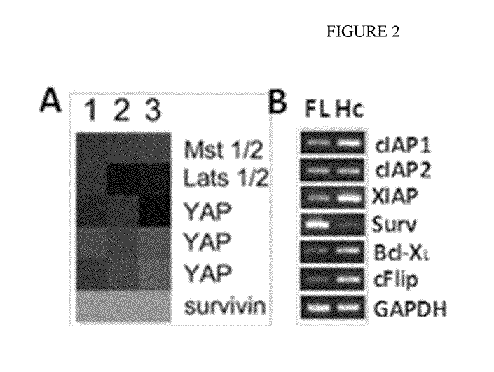

Provided herein are improved methods for the repopulation of host liver hepatocytes by engineering mature normal hepatocytes to express a gene that increases their proliferative potential. Yap, the effector gene of the mammalian Hippo kinase phosphorylation cascade, controls liver size in mice (18). Yap is synthesized on polyribosomes in the cytoplasm and is then transferred to the nucleus, where it complexes with TEA Domain (TEAD) transcription factors and serves as a transcriptional coactivator of many genes, including cell cycle regulating genes that control cell proliferation (FIG. 1; (19, 20). It also induces expression of anti-apoptotic genes Birc2 and Birc5 (survivin) which enhances and prolongs the life-span (survival) of cells by preventing them from undergoing senescence. When Yap is phosphorylated at amino acid S127 by signaling through upstream Hippo pathway kinases, Mst½ and Lats½, it remains in the cytoplasm, is non-functional and is degraded by ubiquination (FIG. 1). We subsequently determined by microarray analysis that fetal liver cells exhibit increased expression of Yap compared to adult liver cells (FIG. 2A), as well as increased expression of Birc5 (survivin) (FIG. 2A), which would further enhance their repopulation potential. Interestingly, Birc5 (survivin) is the only anti-apoptotic gene whose expression is increased in rat fetal liver stem/progenitor cells compared to adult hepatocytes (FIG. 2B).

A major concern following introduction of a gene inducing cell cycle progression is that augmented proliferation of these cells in vivo could lead to tumor formation. When we transplanted rat FLSPC that have much higher proliferative potential than hepatocytes into normal adult rats, we observed a >1000-fold increase in the number of these cells and their progeny in the repopulated liver after 6 months (3). Transplanted cells differentiated into mature hepatocytes, completely integrated into the hepatic tissue structure, made normal junctional contacts with host hepatocytes and showed normal hepatic gene expression (3). There was no hepatic dysplasia or compression of the surrounding hepatic tissue and repopulation continued to increase for up to one year at which time the proliferative activity of the transplanted cells had reduced to the same low level as in host hepatocytes (21). Liver repopulation remained stable for an additional year and there was no evidence for tumor formation by transplanted fetal liver cells or their progeny (21). By contrast, genetically modified mice hyperexpressing Yap in all hepatocytes develop liver hyperplasia and tumorigenesis (18, 22, 23). In addition, human tumors in different organs, including the liver, often show increased Yap expression (19, 24). Accordingly, provided herein are methods that control the expression or function of Yap in transduced cells.

Expression of YAP target genes through controlled activation of YAP according to the methods provided herein increases the proliferative potential of the cells and renders these cells resistant to apoptosis/senescence (FIG. 1). Yap is prevented from functioning by linking it to the estrogen receptor which blocks its function by preventing it from transferring from its site of synthesis in the cytoplasm to its site of function in the nucleus. This system is distinct from the GCSFR-TmR system which uses a genetically modified estrogen receptor to control dimerization of a growth factor receptor to stimulate proliferation of cells (35). In contrast to GCSFR, Yap, is a growth regulator, not a growth factor. In the nucleus, Yap forms a complex with a transcriptional activator gene, TEAD, which leads to expression of Yap target genes that control the cell cycle, increases the proliferative potential of the cells and renders these cells resistant to apoptosis/senescence (see FIG. 1). This further augments the ability of transduced cells to remain viable and replace non-transduced cells in an otherwise normal tissue. As described herein, the YAP-ERT2 system exhibits effective in vivo expansion of cells in the presence of tamoxifen, whereas the GCSFR-TmR system exhibits weak in vivo expansion of transplanted bone marrow cells in response to tamoxifen (49, 50).

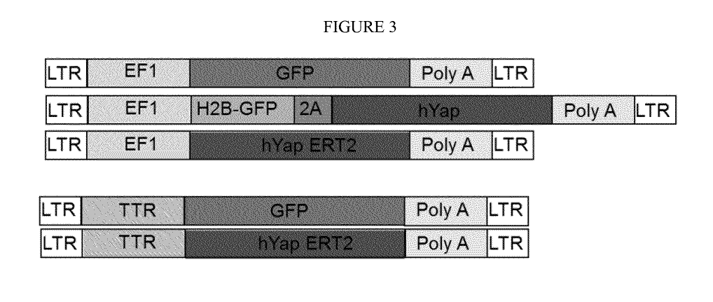

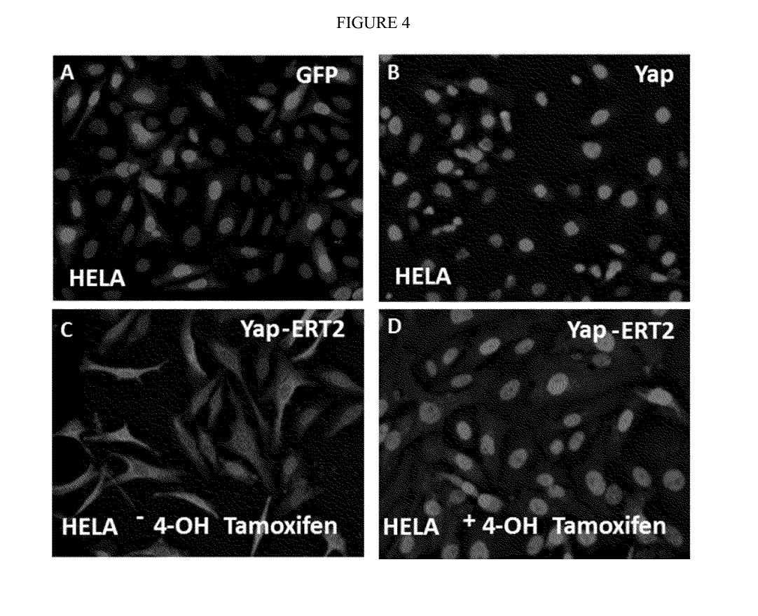

As described in the Examples, one exemplary vector system for introducing the Yap gene into adult hepatocyte is a lentivirus vector system. In this vector system, a human Yap cDNA (hYap) sequence was linked to the ligand binding domain of the estrogen receptor to control Yap function and restrict its oncogenic potential. Several lentivirus vectors were prepared containing Yap, GFP (a control marker gene) or both Yap and GFP under control of a general cellular promoter (EF-1) or the hepatocyte-specific transthyretin (TTR) promoter (FIG. 3). Yap function was further restricted by linking it to a genetically modified ER sequence (ERT2) which is not recognized by native estrogenic hormones (such as 1762 -estradiol) but has very high affinity for the estrogenic hormone analogue, 4-OH tamoxifen, a normal metabolite of tamoxifen (25). As shown in FIG. 4, in the absence of 4-OH tamoxifen, Yap linked to ERT2 is retained in the cytoplasm of HeLa cells in culture. When 4-OH tamoxifen is added to the culture medium at a very low dose (0.1 μM), Yap linked to ERT2 is transferred to the nucleus.

Other Vectors or Delivery Systems to Introduce YAP-ERT2 into Cells or Cell Lines

Although the vector system exemplified in the Examples to introduce YAP-ERT2 into hepatocytes and other cells or cell lines is a third generation lentivirus (26), the methods provided herein are not limited to use of lentivirus vector systems. In some embodiments, alternative viral vectors are used, such as a retrovirus, adenovirus, adeno-associated virus, or Sendai virus (33). In some other embodiments, site-specific integration by homologous recombination is used to introduce genes into cells. In some embodiments, homologous recombination is enhanced by targeted DNA breaks to introduce recombinant DNA sequences (such as YAP-ERT2) into “safe-havens” in the host cellular genome, using zinc finger nuclease (27), talen (28) or CRISPR/cas (29, 30) technologies. In some other embodiments, non-integrating methods of DNA transfection, using an Epstein Barr Nuclear Antigen (EBNA) plasmid, are used to express foreign DNA, such as YAP-ERT2, for up to 6-7 cell divisions (31). In some embodiments, other alternative methods, including self-replicating RNAs (32) are employed to express YAP-ERT2. Any method that allows uptake of nucleic acids into cells, such as through formation of chemical, biochemical, biomatrix, mechanical or electromagnetic complexes with DNA or RNA molecules can be employed to introduce the fusion genes described herein into cells.

The methods provided herein are not limited to transduction/transplantation of primary hepatocytes. In some embodiment, other cells and cell lines, such as embryonic stem (ES) cells and induced pluripotent stem (iPS) cells that can be differentiated along different lineages, including the liver, by genetic manipulation and/or modification of cell culture conditions (34) are used to introduce YAP-ERT2. In some embodiments, expression is controlled under an appropriate promoter that regulates gene transcription in the particular target cell of interest. In certain instances, these methods increase the repopulation potential of these YAP-ERT2 transduced cell lines.

In addition to the liver, the methods provided herein can be applied to the repopulation of other organs and tissues. An example of such an application in another tissue system is the repopulation of the pancreatic islet cells with modified pancreatic islet B-cells that express the YAP-ERT2 fusion protein. In some embodiments, ES cells or iPS cells differentiated toward the insulin producing pancreatic islet β-cell lineage are transduced with the YAP-ERT2 fusion gene under control of the insulin promoter to increase the growth of pancreatic islet B-cells after their inoculation under the kidney capsule or other mode of administration, such as delivery to the liver through splenic injection or portal vein infusion. Such cells will produce and secrete insulin into the circulation and serve as a therapeutic method to treat diabetes. In some embodiments, the therapeutic response is regulated by tamoxifen administration.

### EXAMPLES

### Example 1

### Preparation of a Lentivirus Expressing YAP-ERT2

Preparation of lentivirus Transfer Vectors

All transfer vectors were validated by sequencing.

Plasmid pCCLsin.cPPT.hEF1. GFP. WPRE:

To create a lentivirus transfer vector carrying the GFP gene under control of the EF1 promoter, two different parental plasmids were used: pCCLsin.cPPT.hPGK.GFP.WPRE (providing the vector backbone) and pEF1 GFP (containing the EF1 promoter and GFP sequence in forward orientation). The pCCLsin.cPPT.hPGK.GFP.WPRE plasmid contains unique restriction sites flanking the PGK promoter/GFP fragment; EcoRV at the 5′ end and SalI at the 3′ end. After double digestion, two fragments are produced: vector (6552bp) and PGK/GFP (1275 bp). The vector (6552 bp) was isolated from an agarose gel, blunt-ended and column purified. Because of a lack of matching unique restriction sites in both plasmids, the EF1/GFP sequence was amplified with primers for EF1 and GFP. The vector and EF1/GFP containing fragments were ligated to generate the plasmid pCCLsin.cPPT.hEFLGFP.WPRE. (FIG. 3).

PlasmidpCCLsin.cPPT.hTTR.noORF.WPRE:

The TTR promoter was PCR-amplified from plasmid pRRL_TTR_GFP_240-1, using primer pairs TTR-XhoI-F and TTR-R. The amplified fragment (564bp) was cloned into the SmaI site of pBluescript to generate plasmid pBS-TTR. The primers were designed to contain a unique restriction site to allow easier promoter transfer into different vectors. Plasmid pBS-TTR was used to create a transfer plasmid containing the TTR promoter followed by a short multiple cloning site (MCS), but no gene to be expressed (no ORF). This allows any gene to be inserted after the TTR promoter. The backbone plasmid pCCLsin.cPPT.hPGK.GFP.WPRE contains unique XhoI and SalI restriction sites which flank the PGK promoter and the GFP gene. Using the above mentioned strategy, the two plasmids (pCCLsin.cPPT.hPGK.GFP.WPRE and pBS-TTR) were simultaneously digested with XhoI and SalI enzymes to release the transfer vector and the TTR promoter sequence, respectively. After agarose gel separation and column purification, the two fragments were ligated to create the intermediate TTR transfer vector pCCLsin.cPPT.hTTR.noORF.WPRE.

PlasmidpCCLsin.cPPT.hTTR.GFP.WPRE:

The plasmid pCCLsin.cPPT.hTTR.noORF. WPRE provides 3 unique restriction sites after the TTR promoter (SmaI, EcoRV and SalI). In plasmid pCCLsin.cPPT.hEF 1.GFP.WPRE the target sites for enzymes SmaI and SalI surround the GFP gene. Both plasmids were simultaneously digested with SmaI and SalI; the fragments were gel separated and column purified. pCCLsin.cPPT.hTTR.noORF.WPRE and the GFP gene were then ligated to create the transfer plasmid expressing GFP under the TTR promoter pCCLsin.cPPT.hTTR.GFP.WPRE. (FIG. 3).

PlasmidpCCLsin.cPPT.EF 1.hYap.ERT2.WPRE:

After enzyme digestion with SmaI and SalI, the GFP gene sequence was removed from plasmid pCCLsin.cPPT.hEF 1.GFP.WPRE, which allowed the hYap gene to be inserted. The ORF for the hYap from plasmid P2×Flag CMV2-YAP2 was PCR amplified, using a reverse primer lacking the stop codon. The PCR fragment was digested with SalI and cloned into the aforementioned vector (sticky and blunt end ligation). The final plasmid contained the EF1 promoter followed by the hYap ORF (without a stop codon): pCCLsin.cPPT.hEFI.hYap.noStop.WPRE. The ERT2 sequence was PCR amplified from plasmid pCAG-CreERT2 using primers ER-F and R, containing SalI target sites. The design of the forward primer allows the ERT2 sequence to be attached to the Yap gene in frame, so that both peptides will be synthesized as a single molecule. pCCLsin.cPPT.hEFI.hYap.noStop.WPRE and the amplified ERT2 fragment were digested with SalI, column purified and ligated together to form the transfer plasmid pCCLsin.cPPT.EFI.hYap.ERT2.WPRE. (FIG. 3).

PlasmidpCCLsin.cPPT.EF1.H2B-GFP.2A.hYap.WPRE

The plasmid H2B-GFP was used as a template for amplification of the two linked genes (H2B-GFP), using primers for H2B/SmaI and H2B/2A/SalI. The reverse primer contained the whole 2A sequence (based on plasmid pCX-OKS-2A) together with a SmaI site. The amplified product (1230 bp) was digested with SalI and SmaI, gel separated and column purified. Plasmid pCCLsin.cPPT.hEFLGFP.WPRE was used as a vector donor. The GFP gene was removed after SmaI/SalI digestion and the H2B-GFP-2A fragment was inserted. The hYap sequence was amplified from plasmid P2×Flag CMV2-YAP2 using primers for Yap/Sal-F and Yap/Sal-R which contain a SalI site. The H2B-GFP-2A containing transfer vector and the amplified fragment were digested with SalI, gel-purified and ligated to make the final plasmid used for preparation of pCCLsin.cPPT.EF1.H2B-GFP.2A.hYap.WPRE. (FIG. 3).

PlasmidpCCLsin.cPPT.TTR.hYap.ERT2.WPRE

The plasmid pCCLsin.cPPT.TTR.noORF.WPRE provides 3 unique restriction sites after the TTR promoter (SmaI, EcoRV and SalI). To prepare the transfer vector, this plasmid was digested with SmaI and purified. Using primers Yap-F and ER-R, a DNA fragment containing the Yap gene linked to ERT2 was amplified (2513 bp). Plasmid pCCLsin.cPPT.EFI.hYap.ERT2.WPRE was used as a template and the fragment was gel-purified. Both the blunt end vector and the PCR fragment were ligated to prepare the transfer vector plasmid pCCLsin.cPPT.TTR.hYap.ERT2. WPRE. (FIG. 3).

TABLE 1
Plasmids used to generate lentivirus transfer vectors:

| Plasmid Name | Size | Purpose | Source |
|---|---|---|---|
| pBluescript | 2885 bp | Cloning vector | Stratagene |
| pCAG-CreERT2 | 6783 bp | CreERT2 sequence | Addgene |
| pCX-OKS-2A | 8495 bp | 2A sequence | Addgene |
| P2xFlag CMV2-hYAP2 | 5541 bp | hYAP2 sequence | Gift, Dr. M. Sudol |
| pEF1 | 5388 bp | EF1 promoter sequence | Dr. M. Dabeva |
| H2B-GFP | 5113 bp | H2B-GFP sequence | Addgene |
| pRRL_TTR_GFP_240-1 | 6813 bp | TTR promoter sequence | Gift Dr. A. Follenzi |
| pCCLsin.cPPT.hPGK.GFP.WPRE | 7827 bp | Backbone for all transfer | Gene Therapy Core at |
| | | plasmids | AECOM |

TABLE 2
Primer Sequences used for RT-PCR or qRT-PCR:
RT-PCR primers

| | | Sequence |
|---|---|---|
| Gene | Primer Sequence (rat) | Detected |
| Birc2 (cIAPI) | F 5′-AGCTTGCAAGTGCTGGATTT-3′ (SEQ ID NO: 4) | 359 bp |
| | R 5′-CACCAGGCTCCTACTGAAGC-3′ (SEQ ID NO: 5) | |
| Birc3 (cIAP2) | F 5′-CTAGCCCTCAGCCTCCTCTT-3′ (SEQ ID NO: 6) | 281 bp |
| | R 5′-GCAAAGCAGGCCACTCTATC-3′ (SEQ ID NO: 7) | |
| Bcl-XL | F 5′-ACCGGAGAGCATTCAGTGAT-3′ (SEQ ID NO: 8) | 374 bp |
| | R 5′-GCAGAACTACACCAGCCACA-3′ (SEQ ID NO: 9) | |
| Birc4 (XIAP) | F 5′-GCAGTCCTGTTTCAGCATCA-3′ (SEQ ID NO: 10) | 357 bp |
| | R 5′-GGGTTCCTCGGGTATATGGT-3′ (SEQ ID NO: 11) | |
| Birc5 | F 5′-TAAGCCACTTGTCCCAGCTT-3′ (SEQ ID NO: 12) | 381 bp |
| (survivin) | R 5′-TCCATTACCCCATGGTAGGA-3′ (SEQ ID NO: 13) | |
| cFLAR | F 5′-CATTCACCAGGTGGAGGAGT-3′ (SEQ ID NO: 14) | 346 bp |
| (c-Flip) | R 5′-CGGCCTGTGTAATCCTTTGT-3′ (SEQ ID NO: 15) | |
| GAPDH | F 5′-ATCCACTGGTGCTGCCAAG-3′ (SEQ ID NO: 16) | 371 bp |
| | R 5′-ATGTAGGCCATGAGGTCCAC-3′ (SEQ ID NO: 17) | |

qRT-PCR primers

| | | Sequence |
|---|---|---|
| Gene | | Detected |
| | Primer Sequence (human) | |
| qhBirc5 | F 5′-CAGACTTGGCCCAGTGTTTC-3′ (SEQ ID NO: 18) | 238 bp |
| (survivin) | R 5′-TGCTCGATGGCACGGCGC-3′ (SEQ ID NO: 19) | |
| qhCcnD1 | F 5′-TGCCAACTGGTGTTTGAAAG-3′ (SEQ ID NO: 20) | 102 bp |
| (cyclin D1) | R 5′-CCTTCCGGTGTGAAACATCT-3′ (SEQ ID NO: 21) | |
| qhCtgf | F 5′-TGCCTGCCATTACAACTGTC-3′ (SEQ ID NO: 22) | 84 bp |
| | R 5′-CTTCATGCCATGTCTCCGTA-3′ (SEQ ID NO: 23) | |
| qhMki67 | F 5′-CAGACTCCATGTGCCTGAGA-3′ (SEQ ID NO: 24) | 107 bp |
| | R 5′-TGCACACCTCTTGACACTCC-3′ (SEQ ID NO: 25) | |
| qhSpp1 | F 5′-GCCGAGGTGATAGTGTGGTT-3′ (SEQ ID NO: 26) | 101 bp |
| (osteopontin) | R 5′-TGAGGTGATGTCCTCGTCTG-3′ (SEQ ID NO: 27) | |
| qhGapdh | F 5′-AACAGCGACACCCACTCCTC-3′ (SEQ ID NO: 28) | 85 bp |
| | R5′-CATACCAGGAAATGAGCTTGACAA-3′ (SEQ ID NO: 29) | |
| | Primer Sequence (rat) | |
| qBirc5 | F 5′-CTGATTTGGCCCAGTGTTTT-3′ (SEQ ID NO: 30) | 138 bp |
| (survivin) | R 5′-ACGGTCAGTTCTTCCACCTG-3′ (SEQ ID NO: 31) | |
| qCcnD1 | F 5′-GTGATGGGGTGAAGTTTTGG-3′ (SEQ ID NO: 32) | 81 bp |
| (cyclin D1) | R 5′-CCTCAAAGCCATTCATGTCA-3′ (SEQ ID NO: 33) | |
| qCtgf | F 5′-AGACCTGTGCCTGCCATTAC-3′ (SEQ ID NO: 34) | 92 bp |
| | R 5′-GCTTTACGCCATGTCTCCAT-3′ (SEQ ID NO: 35) | |
| qMki67 | F 5′-ATGCGTCTGCAGAGAAGGTT-3′ (SEQ ID NO: 36) | 101 bp |
| | R 5′-CTGACTTTGCCCAGAGATGA-3′ (SEQ ID NO: 37) | |
| qSpp1 | F 5′-GATCGATAGTGCCGAGAAGC-3′ (SEQ ID NO: 38) | 111 bp |
| (osteopontin) | R 5′-TGAAACTCGTGGCTCTGATG-3′ (SEQ ID NO: 39) | |
| qGapdh | F 5′-GGCATTGCTCTCAATGACAA-3′ (SEQ ID NO: 40) | 95 bp |
| | R 5′-ATGTAGGCCATGAGGTCCAC-3′ (SEQ ID NO: 41) | |

### Example 2

### Characterization of YAP-ERT2 Lentivirus

Materials and Methods

Cell Culture

HEK293T cells were grown in Iscove's modified Dulbecco's medium (IMDM), supplemented with 10% FBS (Hyclone) and L-glutamine (50 U/ml), penicillin and streptomycin (50 U/ml) (Life Technologies, Carlsbad, Calif.). HeLa cells were grown in DMEM/10% FBS and L-glutamine (50 U/ml), penicillin and streptomycin (50 U/ml). Primary rat hepatocytes, isolated by a two-step collagenase perfusion protocol (see below), were initially plated on collagen coated dishes in DMEM/10% FBS until they attached (4-5 hours). The medium was replaced with Block's medium for the duration of the experiments (36).

In Vitro Tamoxifen Treatment

A 10⁻² M stock solution of 4-OH tamoxifen in methanol was stored at −20° C. To prepare a working solution, 4-OH tamoxifen was diluted to 1×10⁻⁷M (0.1 μM) in Blocks medium and applied to cultured cells for 4 days.

Plasmids

A third generation expression system was used to generate lentiviruses by transient transfection of HEK293T cells, using CaPO₄ transfection (37). Four plasmids, provided by the Gene Therapy Core at the Albert Einstein College of Medicine are as follows: pMDLg/pRRE (packaging plasmid containing Gag and Pol), pCMV-VSV-G (envelope plasmid), pRSV-Rev and the self-inactivating (SIN) transfer vector plasmids (based on the backbone of pCCLsin.cPPT.hPGK.GFP.WPRE (see vector design below). The transfer vector plasmids contain a set of genes driven by either the constitutive human elongation factor alpha promoter (EF-1) or the liver specific rat transthyretin promoter (TTR). All additional plasmids used to generate our lentivirus transfer vectors are listed in Table 1.

Virus Production

Virus stocks were produced by calcium phosphate transient transfection, co-transfecting the four plasmids into cultured HEK293T cells (37). The calcium phosphate-DNA precipitate was allowed to remain in contact with the cells for 14-16 h, followed by medium replacement. Cell medium was collected 48 h later, centrifuged at 20,000 rpm for 90 min at room temperature and the pellet (viral particles) was resuspended in DMEM medium (1/200 of the initial volume).

Determination of Viral Titer

The virus particle (VP) concentration was determined by qPCR titration, using the Lenti-X RT-PCR titration kit (Clontech Mountain View, Calif.), according to the manufacturer instructions.

Animals

Inbred male DPP4⁻ F344 rats (8-10 weeks of age) were purchased from Taconic Farms (German Town, N.Y.) and were used as hepatocyte donors. Syngeneic male mutant DPP4⁻ F344 rats (cell transplantation recipients) were provided by the Special Animal Core of the Marion Bessin Liver Research Center at the Albert Einstein College of Medicine.

Isolation of Rat Hepatocytes and Cell Transplantation

Rat livers were perfused with 5 mM EGTA solution, followed by Liberase Blenzyme solution (7 U/100 mL; Roche Applied Science, Indianapolis, Ind.). The livers were excised and minced in DMEM/10% FBS. The cells suspension was filtered through an 80-μm nylon mesh and centrifuged for 2 minutes at 50 g at room temperature (RT). The pellet was washed 3 times, resuspended in DMEM/10% FBS and mixed with an equal volume of Percoll (GE Healthcare Bio-Sciences Corp, Piscataway, N.J.) solution (containing Percoll/10× HBSS, 9:1) and centrifuged for 10 minutes at 50 g at RT. The cell pellet was washed 2 times at 200×g, resuspended in DMEM/10% FBS and used for cell transplantation, ex vivo transduction with lentiviruses or in primary cell culture.

One milliliter of medium containing approximately 5×10⁶ hepatocytes was injected into the recipient's spleen after 2/3 partial hepatectomy, using a 25-gauge needle. All operations were performed under isofluorane anesthesia.

Transduction of Cultured HeLa Cells with Lentiviruses

3×10⁵ HeLa cells/well were plated in six-well culture dishes. The next morning cells were infected with 500 μl/well lentivirus particles with an MOI of 10 (for GFP expressing vectors) resuspended in DMEM/5% FBS. Medium was changed after 24 h and cells were collected after 5 days. 2×10⁵ rat hepatocytes/well were plated in six well culture dishes. After attachment, the cells were infected with 500u1/well lentivirus particles with an MOI of 50 to 100 (for GFP expressing vectors) resuspended in Block's medium. Medium was changed after 24 h and cells were collected 5 days post infection.

Transduction of Isolated Primary Rat Hepatocytes with Lentiviruses

5×10⁶ freshly isolated hepatocytes were washed 2 times with Block's medium at 100×g in a 50 ml tube and the resuspended cells were mixed with lentiviruses not expressing GFP at a level of 500 VP/cell in 500 μl HGM containing 5% HI-FBS, 25 ng/ml EGF and 2×ITS. The cells were incubated at RT for 4 h with gentle agitation. The cells were washed 2 times at 100×g in 40 ml of fresh DMEM/10% FBS and the final pellet was resuspended in 1 ml DMEM/10%FBS. Before transplantation, virally transduced hepatocytes were checked for viability by trypan blue dye exclusion and cells were transplanted only when their viability was or exceeded 80%.

RNA Isolation and Reverse-Transcriptase Polymerase Chain Reaction (RT-PCR). Quantitative PCR

RNA was isolated from cultured cells using Trizol (Life Technologies, Carlsbad, Calif.) according to the manufacturer's instructions and resuspended in RNase free water. RNA was further treated with DNAse I (NEB, Ipswich, Mass.) for 30 min and purified using the RNeasy Mini Kit (Qiagen, Germany).

All reverse transcriptase reactions were carried out with the Verso cDNA Synthesis Kit (Thermo Scientific, Waltham, Mass.), according to the manufacturer's protocol. Rat specific primers for different genes with annealing temperature of 60° C. for all were chosen with the Primer3 program and are presented in Table 2. The expression level of glyceraldehyde-3-phosphate dehydrogenase (GAPDH) was used as an internal control.

For PCR, 20 ng cDNA were mixed with 1× Choice Taq Blue Mastermix (Denville Scientific Inc, Metuchen, N.J.) and 0.5 mM primers and amplified 23 to 33 cycles.

Real time PCR was performed in triplicate for each gene. Each SYBR Green assay was performed in a 12 μl total reaction volume that included 6 μl of 2× SYBR Green Power master mix (Applied Biosystems, Foster City, Calif.), 250 nM of each primer and 20 ng of template cDNA. Assays were run on a 7500FAST instrument (ABI) under standard conditions recommended by the manufacturer and were: 95° C. for 10 min, followed by 40 cycles of 95° C. for 15 sec, and 60° C. for 1 min followed by melting curve analysis. Data were analyzed using 7500 ABI software, V2.0.6. Fold difference in gene expression was determined by the ΔΔCt method.

Tamoxifen Administration In Vivo

Grain based rodent chow pellets containing 400-500 mg/Kg tamoxifen citrate (Sigma) was purchased from Bio-Serv, Flemington, N.J. and fed ad lib to rats immediately following transplantation of lenti TTR-YapERT2 transduced hepatocytes, lenti TTR-GFP transduced hepatocytes or mock transduced hepatocytes and continued as indicated in the various studies.

DPP4 Enzyme Histochemistry

5 μm cryosections were air dried and fixed in 95% ethanol-glacial acetic acid (99:1 vol/vol) for 5 min, followed by a wash step in cold 95% ethanol for 5 min. Sections were air dried and incubated at 37° C. for 45 min in 100m1 substrate solution containing 100 mg Fast blue BB salt (Sigma), 50 mg glycyl-propinr-4-methoxy-b-naphtylamide (GPMN) (Sigma) dissolved in TMS buffer (0.1 m Trisma Maleate /0.1M NaCl; pH6.5). Following washing ×3 in 0.4M NaCl, the sections were incubated in 0.1M CuSO₄ for 10 min, washed 2 times with water, 10 min each, fixed in 4% PFA (Sigma) in PBS for 10 min and rinsed with water. Liver sections were counterstained with Hematoxylin for 20 seconds, rinsed with water and air dried.

Immunofluorescence Microscopy of Liver Tissue Sections

Five micron sections of frozen liver tissue were fixed in 4% PFA in PBS for 10 min at room temperature and treated with sodium borohydride. For detection of cytoplasmic proteins, the sections were permeabilized with 0.3% Triton X-100. Blocking was with 5% normal serum from the animal species in which the secondary antibody was raised and 2% bovine serum albumin (BSA). Primary antibodies were applied overnight at 4° C. in 2% normal serum/2% BSA. The secondary, fluorescent conjugated antibody was applied for 40 min at room temperature. Sections were counterstained with 4′,6-diamidino-2-phenylindole (DAPI). Fluorescence images were obtained with a Nikon Eclipse TE 2000-S fluorescence microscope. Primary and secondary antibodies used in the different IF analyses are given in the Table 3.

TABLE 3
Antibodies used to detect specific proteins in repopulating clusters of
lenti TTR-Yap-ERT2-transduced DPPIV⁺ hepatocytes transplanted into DPPIV⁻ rat
recipient liver

| | | | |
|---|---|---|---|
| Antibodies | Company or Producer | Cat. Number | Dilution |

Primary antibodies

| | | | | |
|---|---|---|---|---|
| | Isotype | | | |
| Mouse anti-rat | IgG1 | Santa Cruz Biotech. | sc-52642 | 1:50 |
| CD26/DPP4 | | | | |
| Rabbit anti- | Polyclonal | Gift, Dr. N. Roy- | | 1:100 |
| human albumin | | Chowdhury | | |
| Rabbit anti- | Polyclonal | Santa Cruz Biotech | sc-8987 | 1:50 |
| human HNF-4a | | | | |
| Rabbit anti-rat | Polyclonal | Gift, Dr. R. Stockert | | 1:100 |
| ASGPR | | | | |
| Rabbit anti- | Polyclonal | Novus Biologicals | NB110-58358 | 1:50 |
| human Yap1 | | | | |
| Mouse anti- | IgG1 | BD Pharmingen | 550609 | 1:50 |
| human Ki-67 | | | | |
| Mouse anti- | IgG1 | Novocastra Laboratories Ltd. | NCL-CK19 | 1:100 |
| human CK19 | | | | |
| Rabbit anti-Sox9 | Polyclonal | EMD Millipore | AB5535 | 1:50 |
| Mouse anti-rat | IgG1 | BioVendor, LLC | RD-680 | 1:75 |
| EpCAM | | | | |
| Rabbit anti- | Polyclonal | Thermo Fisher Scientific | RB-365-A1 | 1:100 |
| human Afp | | | | |
| Mouse anti-rat | IgG1 | Gift, Dr. S. Sell | | 1:50 |
| OV6 | | | | |
| Rabbit anti- | Polyclonal | ABCAM | ab19898 | 1:25 |
| CD133 | | | | |
| Mouse anti-rat | IgG2a | Cedarlane | CL044 | 1:50 |
| CD44 | | | | |
| Mouse anti-rat | IgG2a | Cedarlane | CL061AP | 1:50 |
| CD26/DPP4 | | | | |

Secondary fluorescent antibodies

| | | | | |
|---|---|---|---|---|
| | Fluorofore | | | |
| Donkey anti-mouse IgG | DyLight 549 | Jackson | # 715-505- | 1:400 |
| | | Immunoresearch | 151 | |
| Goat anti-mouse IgG1 | cy3 | Same | #715-165- | 1:400 |
| | conjugated | | 151 | |
| Goat anti-mouse IgG2a | cy3 | Same | #115-165- | 1:400 |
| | conjugated | | 206 | |
| Goat anti-mouse IgG | cy2 | Same | #115-225- | 1:400 |
| | conjugated | | 205 | |
| Donkey anti-rabbit IgG | DyLight 488 | Same | #715-095- | 1:200 |
| | | | 151 | |
| Donkey anti-rabbit IgG | Cy3 | Same | #711-167- | 1:200 |
| | conjugated | | 003 | |

Quantification of Liver Repopulation By DPPIV⁺ Hepatocytes in DPPIV⁻ Recipients

5 μm frozen liver sections stained for DPPIV expression were imaged using a Zeiss AxioObserver microscope with the 5×, 0.16 NA objective and images were captured with a Zeiss Axiocam HRc color camera. Sequential images were mapped and stitched together using Zeiss Axiovision software (version 4.8). Total area and DPPIV positive area of liver sections were quantified using ImageJ software (Rasband, W. S., ImageJ, U.S. National Institutes of Health, Bethesda, Md., USA, http://imagej.nih.gov/ij/, 1997-2014). Color images were segmented using Lab Color Space. The lightness (L*) dimension was used to threshold the total area, and the b* color-opponent dimension was used to threshold orange, DPPIV positive cells. The ratio of these measurements gave the percent repopulation. Statistics were calculated using Microsoft Excel.

Results:

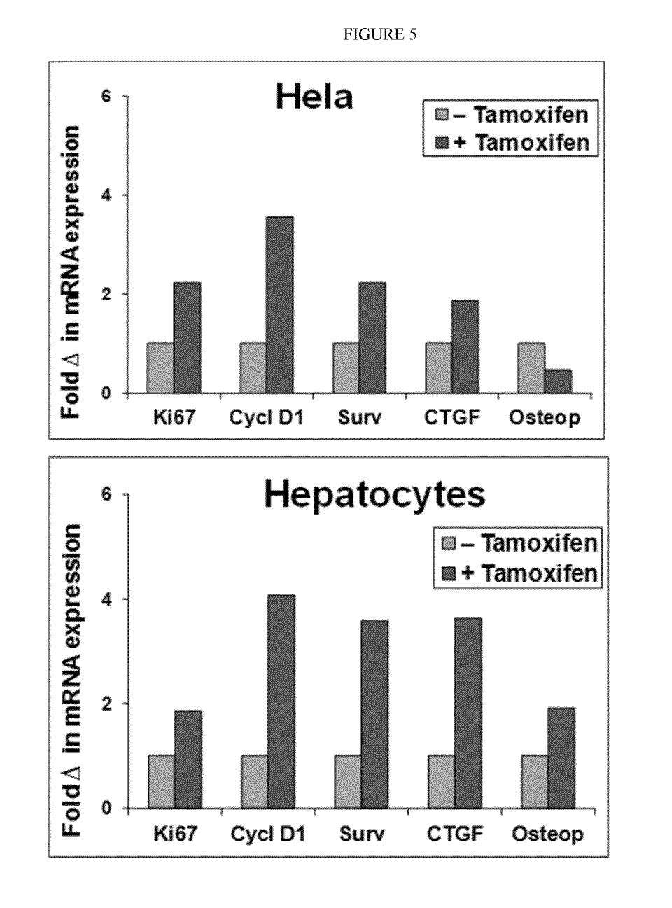

We first tested a lentivirus EF1-hYAP-ERT2 vector in cultured HeLa cells and demonstrated that after transduction of HeLa cells with this virus, hYap synthesized from the transduced gene (containing a flag tag at its 5′ end, so that it could be detected by immunohistochemistry in hYAP-ERT2 transduced cells) was retained in the cytoplasm in the absence of tamoxifen but was transferred to the nucleus upon tamoxifen administration (FIG. 4). We subsequently demonstrated a tamoxifen dependent increase in expression of mRNAs for several well established Yap target genes, including Ki67, cyclin D1, survivin and CTGF, in both HeLa cells and rat hepatocytes transduced with lenti EF1-YAP-ERT2 (FIG. 5). Thus, we established two features of the method; namely, 1) control of the subcellular location of Yap through linkage to the estrogen receptor and tamoxifen administration (FIGS. 4) and 2) nuclear function of YAP-ERT2 as a transcriptional coactivator in transduced hepatocytes when the cells were cultured in the presence of 0.1 uM tamoxifen (FIG. 5).

For in vivo studies, we placed hYAP-ERT2 under control of the transthyretin promoter (TTR), so that its expression would be tightly controlled and occur only in hepatocytes (38). This would eliminate the possibility that small numbers of non-hepatic cells in isolated hepatocyte preparations could be transduced and potentially responsible for liver repopulation. In a previous study, isolated hepatocytes were incubated in suspension with lenti CMV-GFP for 4 hours and then transplanted into the liver of normal mice in conjunction with 2/3 partial hepatectomy (26). Using 2×10⁷ lenti CMV GFP transduced hepatocytes, isolated GFP⁺ cells and very small clusters of GFP⁺ cells were identified at 2 or 5 weeks but repopulation levels were very low (0.4-1.0%).

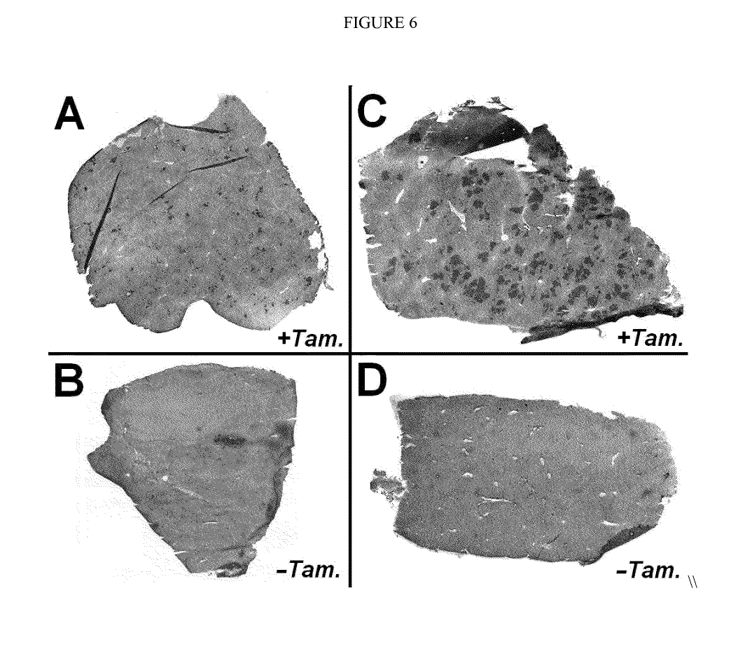

Using a comparable transduction protocol, we transplanted 5×10⁶ lenti TTR-YAP-ERT2 transduced hepatocytes. The donor hepatocytes were from inbred Fischer 344 rats that are WT for a cell surface marker gene, dipeptidylpeptiase IV (DPPIV⁺), whereas recipients are Fischer 344 rats that have a natural mutation in the DPPIV gene and are negative for DPPIV expression (DPPIV⁻). In this model system, transduced cells and their progeny can be readily detected by DPPIV enzyme histochemistry (39,40). The liver phenotype of DPPIV⁻ rats is otherwise normal (40). Studies illustrated in FIG. 6 demonstrated that lenti TTR-hYAP-ERT2 transduced hepatocytes repopulate the normal adult liver in rats fed tamoxifen and repopulation was nil in the absence of tamoxifen feeding.

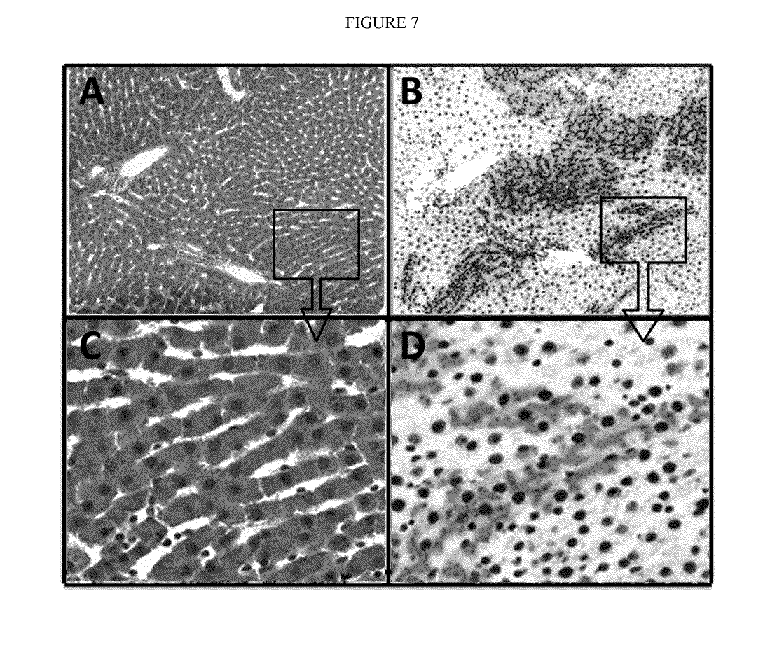

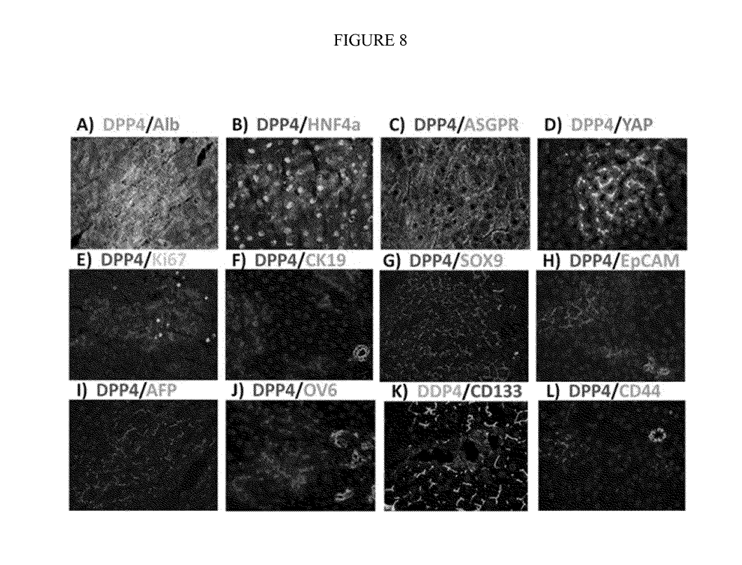

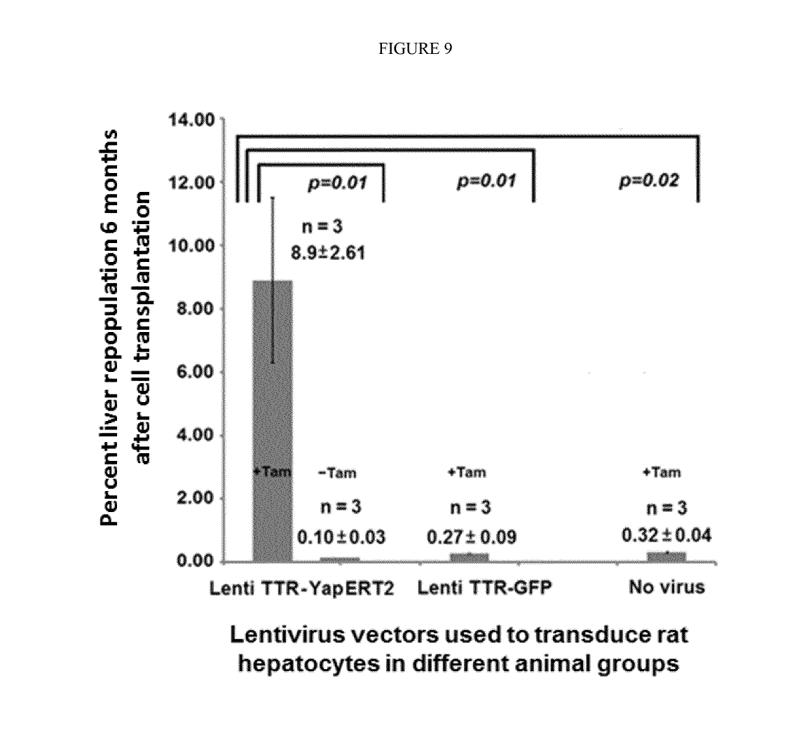

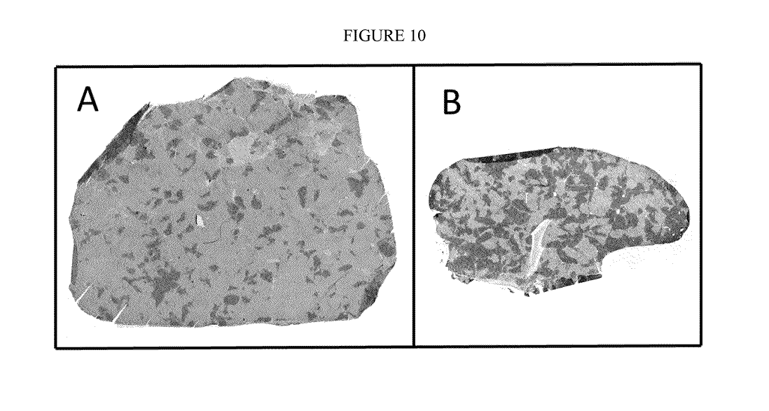

These studies used ¼ the number of transplanted hepatocytes as used previously (26) and established a third critical feature of the method, namely, that we can regulate the growth and repopulation potential of hYAP-ERT2 transduced hepatocytes in vivo by tamoxifen administration. In the repopulated normal rat liver, transplanted lenti TTR-hYAP-ERT2 transduced hepatocytes and their progeny are morphologically normal at 6 mos. after transplantation and have become fully incorporated into the hepatic parenchymal plates (FIG. 7). The repopulating cells have the gene expression pattern of differentiated hepatocytes (i.e., they express albumin, HNF4a and ASGPR) without evidence of dedifferentiation, cellular dysplasia or expression of liver progenitor or cancer stem cell genes (i.e., they do not express AFP, OV6, CK19, Sox9, CD133 or CD44) (FIG. 8). Repopulation with lenti TTR-hYAP-ERT2 transduced hepatocytes was 8.90%±2.64% SEM at 6 mos after cell transplantation in the presence of tamoxifen feeding (+tam) and 0.01%±0.03% SEM in the absence of tamoxifen feeding (−tam) P=0.01, was 0.27%±0.09% SEM with lenti TTR-GFP in tamoxifen fed rats (+tam) P=0.01 and 0.32%±0.04% SEM in non-virally transduced hepatocytes also under the same conditions P=0.02 (FIG. 9). At one year after transplantation of lenti TTR-hYAP-ERT2 transduced hepatocytes, we have observed 20-50% repopulation of the host liver, the transplanted hepatocytes appear normal morphologically, the liver structure is normal and there is no evidence of hepatocyte malignancy (FIG. 10).

It is generally agreed that the amount of cell replacement required to achieve effective therapy in inherited metabolic disorders of the liver, such as Crigler-Najjar Syndrome, Type 1 (CN1), Familial Hypercholesterolemia, phenylketonuria, Factor IX deficiency, ornithine transcarbamylase deficiency, etc.) is 3-5% (41-43) and several studies have reported an initial therapeutic response when hepatocytes comprising 1-5% of total hepatocytic mass were transplanted into patients with CN1 (43,44) and Familial Hypercholesterolemia (45). The problem is that in all instances, the transplanted hepatocytes did not proliferate after their engraftment into the liver (34, 42-45). The therapeutic response was only temporary and the most likely explanation is that the transplanted cells were lost over time either by normal liver cell turnover or immunorejection (34). If transplanted hepatocytes could be induced to undergo 5-6 divisions by transduction with a vector stimulating their proliferation in a regulatable fashion, such as with lenti TTT-hYAP-ERT2/tamoxifen, we could increase the number of transplanted hepatocytes repopulating the liver by about 50 fold, thereby increasing potential effectiveness of therapy, as well as reducing the number of cells needed for therapy in a given patient. We have overcome low repopulation and loss of transplanted hepatocytes by engineering donor cells to exhibit and retain augmented proliferative potential by introducing the proliferative gene hYap linked to ERT2 with function controlled by tamoxifen. Moreover, since lentivirus transgene integration into the host genome is stable, proliferation of transplanted lenti-TTR-hYAP-ERT2 transduced hepatocytes can be reactivated at any time by tamoxifen administration to boost cell replacement as we monitor patients over time for maintenance of effective therapy. Thus, we anticipate achieving life-long therapy with the methods we have developed. Since the transduced hepatocytes with augmented proliferative potential we have used for repopulation in animal model studies are derived from normal (WT) donors, use of this single vector will be applicable for treatment of patients with a wide variety of inherited metabolic disorders and potentially other liver diseases in which there is a loss of hepatic function, such as in hepatic fibrosis/cirrhosis. Examples for treatment of an animal model for a human inherited metabolic disorder (the Gunn rat) and hepatic fibrosis/cirrhosis (DPPIV⁻ F344 rat treated with thioacetamide (TAA) for 10-12 weeks), using lentivirus TTR-hYAP-ERT2 transduced hepatocytes to repopulate the diseased liver, are given below.

### Example 3

### Treatment of a Genetic-Based liver Disease By Liver Repopulation with Lenti TTR-hYap-ERT2 Transduced Hepatocytes in an Animal Model of Hyperbilirubinemia

The Gunn rat is an animal model for Crigler-Najjar Syndrome Type 1 (CN1), a genetic-based liver disease in which mutations in the UDP-glucuronosyl transferase gene cause either inactivation of gene expression, truncation of the protein or loss of enzyme activity, leading to kernicterus, brain damage and mental retardation (11,42,43). It has been estimated that increased serum bilirubin levels in CN1 Syndrome could be cured if 3%-5% of liver mass was replaced by transplanted hepatocytes with normal bilirubin metabolism (42,43) and two previous studies have reported a decrease in serum bilirubin in several patients with CN1 syndrome by transplantation of normal hepatocytes (43,44). However, the therapeutic effect was only temporary, because the transplanted hepatocytes did not proliferate and were gradually lost by either normal cell turnover or immunorejection.

Experiments will be conducted in Gunn rats transplanted with lenti TTR-YapERT2 transduced WT hepatocytes to test both the effectiveness and durability of therapy (Protocol 1) and the ability to reinitiate and modulate the therapeutic response by on/off cycles of tamoxifen administration (Protocol 2). We hypothesize that transplantation of lenti TTR-hYAP-ERT2 transduced hepatocytes from congenic (WT) Wistar-RHA rats into Gunn rats of the same genetic background will produce a progressive reduction in serum bilirubin from an initial level of ˜7-8 mg/dl to less than 1 mg/dl (a normal level) when tamoxifen at a dose of 400-500 mg/kg is included in the feed. This is based on our expectation that lenti TTR-hYAP-ERT2 transduced WT hepatocytes will progressively replace host Gunn rat hepatocytes under tamoxifen administration, similar to results we have obtained in the DPPIV hepatocyte transplantation model. Gunn rats transplanted with lenti TTR-hYAP-ERT2 transduced WT hepatocytes, but not treated with tamoxifen feed, will exhibit a modest (25-30%) reduction in serum bilirubin, because the transplanted WT hepatocytes express the UGT-1A1 gene and will replace ˜0.5% of hepatocyte mass but will not expand in the absence of tamoxifen administration.

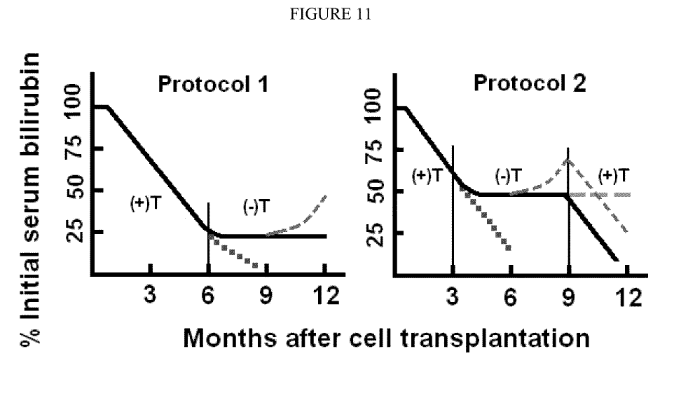

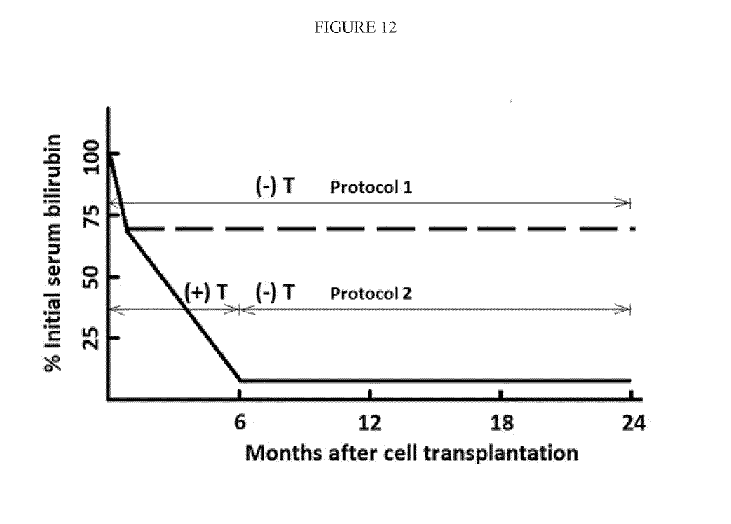

In our standard protocol, 5×10⁶ virally transduced WT hepatocytes are transplanted into recipients with a 5%-10% engraftment efficiency in which the host liver contains ˜150-200×10⁶ hepatocytes after partial hepatectomy. Initially, we will monitor serum bilirubin levels weekly as a measure of liver cell replacement/repopulation by transplanted lenti TTR-hYAP-ERT2 transduced hepatocytes (see FIG. 11). We expect serum bilirubin to be reduced progressively with ˜50% reduction at 3 months. In Protocol 1, tamoxifen feeding will be continued until serum bilirubin is reduced by ˜75%-80% (at ˜5-6 months during tamoxifen feeding). Animals will then be switched to normal rat chow and serum bilirubin monitored for the next 6 months. It is anticipated that serum bilirubin will continue to decrease until tamoxifen is cleared from host tissue stores (˜1 month) and will then remain stable. However, if proliferation of transplanted lenti TTR-hYAP-ERT2 hepatocytes has escaped from tamoxifen control, serum bilirubin will continue to decrease ( [symbol] ). On the other hand, if non-proliferating lenti TTR-hYAP-ERT2 transduced hepatocytes are lost from the host tissue, serum bilirubin levels will increase during the withdrawal period ( [symbol] ). These studies will demonstrate the effectiveness of lenti TTR-hYAP-ERT2 transduced hepatocytes in treating hyperbilirubinemia in the Gunn rat model. Liver repopulation at various times after cell transplantation will be quantified by sacrificing individual rats and determining the % liver repopulation by immunohistochemistry for UGT-1A1 positive cells and measuring UGT-1A1 enzyme activity by biochemical analysis in liver homogenates, using standard protocols developed by our colleague, Dr. Jayanta Roy-Chowdhury (46). The levels of serum bilirubin vs. the % hepatocyte replacement will be plotted to determine whether there is a direct correlation.

In Protocol 2, a similar experiment will be conducted, but tamoxifen will be discontinued when serum bilirubin is reduced by ˜50%. Gunn rats with transplanted virally transduced hepatocytes will then be maintained on a normal diet for 6 months. If the serum bilirubin stabilizes or increases during this period, tamoxifen feed will be restarted to determine whether there is a subsequent reduction in serum bilirubin. If the latter occurs, this will indicate that the virally transduced hepatocytes have retained their potential for repopulating the liver during and after the period of tamoxifen withdrawal. The results of this experiment will have very significant implications concerning the effectiveness of our vector system for long-term therapeutic hepatocyte repopulation.

### Example 4

### Treatment of Hyperbilirubinemia in the Gunn Rat By a Combination of Cell and Gene Therapy.

For clinical applications in humans with genetic disorders of the liver, it would be most advantageous to transplant hepatocytes isolated from a patient back into the same patient after the genetic defect has been corrected in vitro by gene therapy. This would avoid the need for immunosuppressive therapy in the recipient. Such studies have been conducted in Gunn rats in which isolated hepatocytes from one Gunn rat were transduced with a lentivirus containing the UGT-1A1 gene (deficient in this animal strain) and then immediately transplanted into a second Gunn rat (47). In this study, there was a 30% reduction in serum bilirubin associated with 0.5-1.0% liver repopulation, which was stable for 8 mo. Because of limitations in the number of hepatocytes that can be transplanted in a single session without causing major liver damage or death of the recipient (43, 45), this is the maximum level of repopulation that can be achieved, because the transplanted cells do not have a selective advantage over host hepatocytes. As noted previously, to achieve complete amelioration of hyperbilirubinemia in Gunn rats or in patients with CN Syndrome, Type 1 will require 3-5% liver repopulation (41-43).

Based on our results using lenti TTR-YapERT2 transduced hepatocytes in the DPPIV model (FIGS. 6-9), and expected results in Example 1 using lenti TTR-YapERT2 transduced normal hepatocytes under tamoxifen control in the Gunn rat model (FIG. 11), we will transduce Gunn rat hepatocytes simultaneously with lenti TTR-YapERT2 and lenti EF1-UGT-1A1 (or under another ubiquitous promoter) and transplant the doubly transduced hepatocytes into a second Gunn rat using our standard protocol (see Materials and Methods). In the absence of tamoxifen administration (Protocol 1), we expect no more than a 30% reduction in serum bilirubin, as obtained previously with Gunn rat hepatocytes transduced with only the EF1 UGT-1A1 gene (47). This is because the lenti TTR-Yap ERT2 gene will be inactive in the absence of tamoxifen administration and the cells will not expand. In Protocol 2, in the presence of tamoxifen administration the cells will expand and we expect serum bilirubin to be reduced to normal by 6 mo. When serum bilirubin is reduced to normal levels (in Gunn rats transplanted with doubly transduced hepatocytes under tamoxifen administration), tamoxifen feeding will be discontinued. The rats will be monitored for serum bilirubin for up to two years (the lifespan of the Gunn rat) with the expectation that serum bilirubin will remain normal. If serum bilirubin rises, tamoxifen treatment will be restarted and we expect serum bilirubin to quickly return to normal. These studies will further demonstrate the specificity and durability of our hepatocyte transplantation protocols and their potential use in humans with genetic disorders of the liver in which there is no underlying liver injury or damage.

### Example 5

### Repopulation of the Fibrotic/Cirrhotic Rat Liver By lenti TTR-hYAP-ERT2 Transduced Hepatocytes

In a previous study (48), we reported that fetal liver stem/progenitor cells (FLSPC) can repopulate the fibrotic/cirrhotic liver to a level of 35-40% at 2-4 months after cell transplantation. With adult hepatocytes, repopulation was much less, i.e., 8-10%. With FLSPC, ongoing fibrogenesis was ameliorated by cell transplantation and there was a modest reduction in fibrosis (48). Since a higher level of hepatocyte replacement will be necessary to restore liver function in fibrotic/cirrhotic patients, as compared to correcting a genetic based liver disorder in which there no underlying or ongoing liver injury/hepatic fibrosis, we reasoned that using lenti TTR-hYAP-ERT2 transduced WT hepatocytes for repopulation will be superior to untreated WT hepatocytes. If we can achieve 20-25% liver repopulation by lenti TTR-hYAP-ERT2 transduced hepatocytes in an animal model of hepatic fibrosis/cirrhosis, this will produce a significant therapeutic effect in terms of liver function, if not also an amelioration of fibrosis.

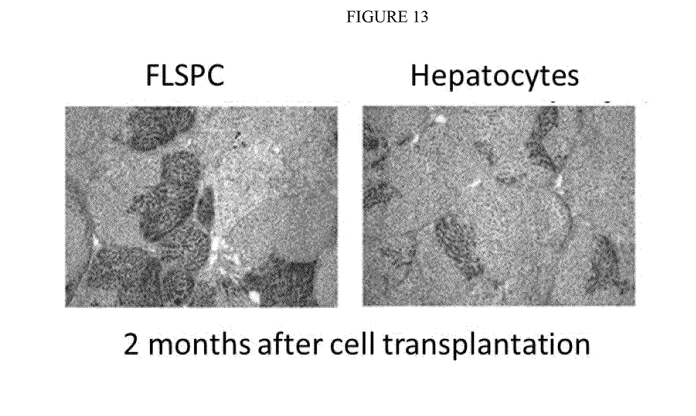

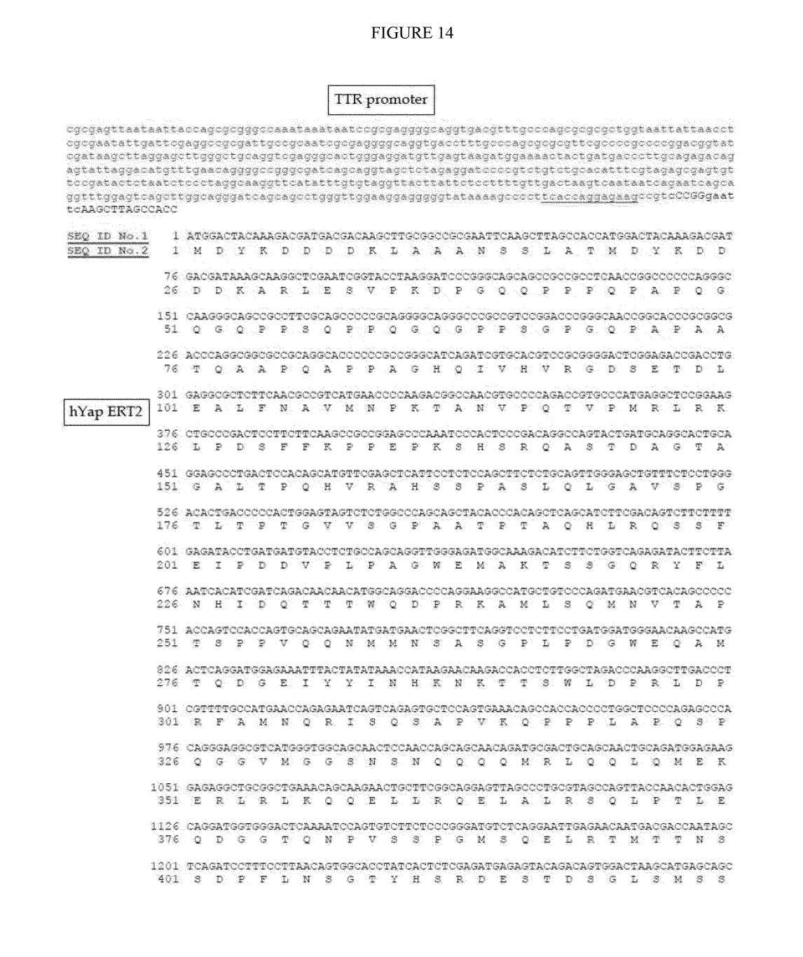

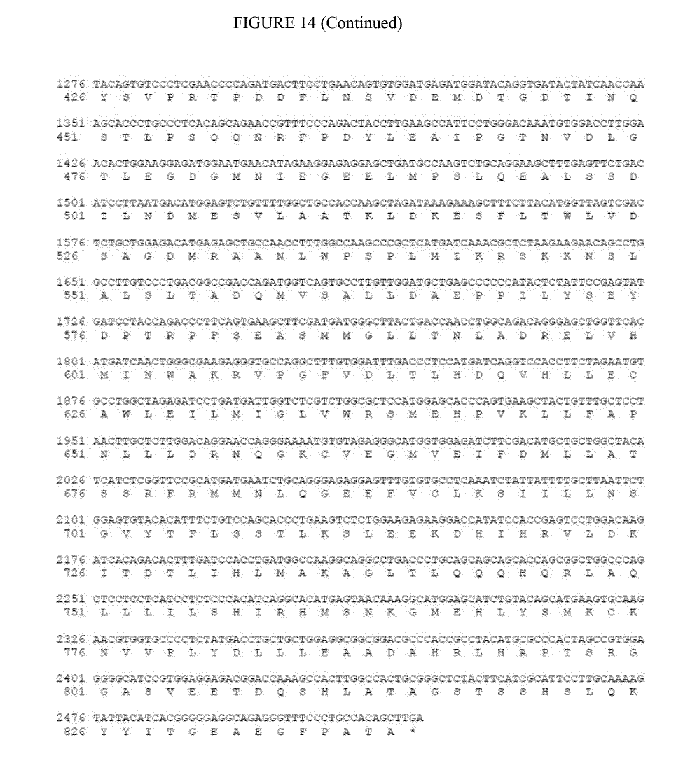

In these studies, we will use the TAA model for hepatic fibrosis/cirrhosis, which we have used previously (48) and is the animal model that most closely resembles human hepatic fibrosis/cirrhosis. After 10-12 weeks of TAA administration, 200 mg/kg body weight IP, 5×10⁶ lenti TTR-hYAP-ERT2 transduced DPPIV⁺ hepatocytes will be transplanted vs. non transduced hepatocytes into DPPIV⁻ rats and TAA will be discontinued. Animals will be followed for up to 4 mos. The level of liver repopulation will be measured monthly together with determination of standard liver function tests and tissue analysis for expression fibrogenesis genes, histochemical analysis of fibrogenesis and levels of fibrosis in animals treated with lenti TTR-hYAP-ERT2 transduced hepatocytes, WT hepatocytes and untreated controls. The detailed experimental plan will be comparable to our previous study (48), except that TAA will be discontinued during the period of cell therapy. This will simplify analysis of the data. Examples from our previous study (48) of the level of liver repopulation by transplanted FLSPC vs. adult hepatocytes at 2 mos. after cell transplantation are shown in FIG. 13. We expect that transplantation of lenti TTR-hYAP-ERT2 transduced hepatocytes will increase repopulation by 2-3 fold compared to untreated hepatocytes (˜25-30% repopulation), and there will also be a clear reduction in fibrosis in cell transplanted vs. control animals.

In other studies, we will combine transplantation of lenti TTR-YapERT2 transduced hepatocytes with treatment of fibrotic/cirrhotic rats with antifibrotic agents. In our previous study (48), we reported a decrease in fibrogenesis and hepatic stellate cell numbers in fibrotic/cirrhotic rats transplanted with FLSPC, and we expect a synergistic response between increased liver regeneration/repopulation by transplanted hepatocytes and reduced fibrosis in fibrotic/cirrhotic rats treated simultaneously with lenti TTR-YapERT2 transduced hepatocytes and an antifibrotic agent, such as, but not limited to, Sorafemid, largazole, galectin inhibitors, FG-3019 (an anti-CTGF antibody), Pirfenidone, a TGF-β inhibitor, endostatin peptide and Polarezin. This will lead to increased liver function and survival in such treated animals and to development of a potentially effective treatment in humans with hepatic fibrosis/cirrhosis of various etiologies.

### SUMMARY

We have constructed a lentivirus containing the proliferative gene, Yap, through which we have successfully transduced hepatocytes in suspension, transplanted these hepatocytes into normal adult rat liver and repopulated the liver to a level sufficient to achieve effective cell therapy in various genetic-based metabolic disorders of liver function. Function of the transduced Yap gene is controlled by linking it to the estrogen receptor through which we can modulate its nuclear vs. cytoplasmic location by administration of tamoxifen, a specific ligand for the estrogen receptor used in this vector (ERT2). This allows us to control the proliferation of virally transduced cells in vivo, thereby restricting their oncogenic potential. Expression of the transduced YAP-ERT2 gene is also under control of the hepatocyte-specific TTR promoter, which eliminates the possibility of Yap expression in small numbers of non-hepatocytes which may be present in our isolated liver cell preparations and could spread to or seed other organs in the body after cell transplantation. This further limits the oncogenic potential of our cell therapy protocol. If the therapeutic effect following cell therapy becomes diminished over time because of loss of transplanted cells, repopulation can be re-induced by retreating cell recipients with tamoxifen. This will preserve the longevity of therapeutic liver repopulation. A final consideration in the innovative and unique features of methods disclosed herein is that the same lentivirus vector can be used to treat a large variety of genetic-based and non-genetic based liver diseases in which there is loss of functioning liver tissue, because the cells in which the gene is introduced are normal, fully differentiated hepatocytes. These cells have a stable phenotype as compared to ES, iPS or other genetically modified or reprogrammed progenitor cells in which the phenotype may be less stable. This is an additional safety feature favoring the use of well-differentiated tissue specific cells for organ repopulation. The specific vector system used for introducing the YAP-ERT2 sequence into primary hepatocytes (or hepatocyte cell lines that may be developed in the future) is not limited to a lentivirus, as YAP-ERT2 can be incorporated into other vector systems to introduce this transgene into mammalian cells. Finally, it should be emphasized that the liver is an excellent solid organ candidate to be used for tissue repopulation because of its portal circulation that is conducive to uniform seeding of the tissue with transplanted cells, its high regenerative (and remodeling) capacity and the ability of transplanted cells to be fully integrated into the normal liver structure.

The examples and embodiments described herein are for illustrative purposes only and various modifications or changes suggested to persons skilled in the art are to be included within the spirit and purview of this application and scope of the appended claims.

### REFERENCES CITED

1. Perera, M. T., Mirza, D. F., and Elias, E. 2009. Liver transplantation: Issues for the next 20 years. J Gastroenterol Hepatol 24 Suppl 3:S124-131.
2. Sandhu, J. S., Petkov, P. M., Dabeva, M. D., and Shafritz, D. A. 2001. Stem cell properties and repopulation of the rat liver by fetal liver epithelial progenitor cells. Am J Pathol 159:1323-1334.
3. Oertel, M., Menthena, A., Dabeva, M. D., and Shafritz, D. A. 2006. Cell competition leads to a high level of normal liver reconstitution by transplanted fetal liver stem/progenitor cells. i 130:507-520; quiz 590.
4. de la Cova, C., Abril, M., Bellosta, P., Gallant, P., and Johnston, L. A. 2004. Drosophila myc regulates organ size by inducing cell competition. Cell 117:107-116.
5. Moreno, E., and Basler, K. 2004. dMyc transforms cells into super-competitors. Cell 117:117-129.
6. Rhim, J. A., Sandgren, E. P., Degen, J. L., Palmiter, R. D., and Brinster, R. L. 1994. Replacement of diseased mouse liver by hepatic cell transplantation. Science 263:1149-1152.
7. Overturf, K., Al-Dhalimy, M., Tanguay, R., Brantly, M., Ou, C. N., Finegold, M., and Grompe, M. 1996. Hepatocytes corrected by gene therapy are selected in vivo in a murine model of hereditary tyrosinaemia type I. Nat Gen 12:266-273.
8. Laconi, E., Oren, R., Mukhopadhyay, D. K., Hurston, E., Laconi, S., Pani, P., Dabeva, M. D., and Shafritz, D. A. 1998. Long-term, near-total liver replacement by transplantation of isolated hepatocytes in rats treated with retrorsine. Am J Pathol 153:319-329.
9. Witek, R. P., Fisher, S. H., and Petersen, B. E. 2005. Monocrotaline, an alternative to retrorsine-based hepatocyte transplantation in rodents. Cell Transplantation 14:41-47.
10. Guha, C., Sharma, A., Gupta, S., Alfieri, A., Gorla, G. R., Gagandeep, S., Sokhi, R., Roy-Chowdhury, N., Tanaka, K. E., Vikram, B., et al. 1999. Amelioration of radiation-induced liver damage in partially hepatectomized rats by hepatocyte transplantation. Cancer Research 59:5871-5874.
11. Chowdhury J R, W. A., Chowdhury N R, Arias I M. 2001. Hereditary jaundice and disorders of bilirubin metabolism. In The Metabolic and Molecular Bases of Inherited Disease. B. A. Scriver C R, Sly W S, Valle D., editor. New York: McGraw-Hill. 3063-3101
12. Finkelstein, J. E., Hauser, E. R., Leonard, C. O., and Brusilow, S. W. 1990. Late-onset ornithine transcarbamylase deficiency in male patients. J Pediatr 117:897-902.
13. Goldstein J L, H. H., Brown M S. 2001. Familial hypercholesterolemia. In The Metabolic and Molecular Bases of Inherited Disease. B. A. Scriver C R, Sly W S, Valle D., editor. New York: McGraw-Hill. 2863-2913.
14. Kaufman, R. J., and Powell, J. S. 2013. Molecular approaches for improved clotting factors for hemophilia. Hematology Am Soc Hematol Educ Program 2013:30-36.
15. Leumann, E., and Hoppe, B. 2001. The primary hyperoxalurias. J Am Soc Nephrol 12:1986-1993.
16. Michalopoulos, G. K., and DeFrances, M. C. 1997. Liver regeneration. Science 276:60-66.
17. Gupta, S., Chowdhury, N. R., Jagtiani, R., Gustin, K., Aragona, E., Shafritz, D. A., Chowdhury, J. R., and Burk, R. D. 1990. A novel system for transplantation of isolated hepatocytes utilizing HBsAg-producing transgenic donor cells. Transplantation 50:472-475.
18. Dong, J., Feldmann, G., Huang, J., Wu, S., Zhang, N., Comerford, S. A., Gayyed, M. F., Anders, R. A., Maitra, A., and Pan, D. 2007. Elucidation of a universal size-control mechanism in Drosophila and mammals. Cell 130:1120-1133.
19. Pan, D. 2010. The hippo signaling pathway in development and cancer. Dev Cell 19:491-505.
20. Zhao, B., Li, L., Lei, Q., and Guan, K. L. 2010. The Hippo-YAP pathway in organ size control and tumorigenesis: an updated version. Genes and Development 24:862-874.
21. Shafritz, D. A., and Oertel, M. 2011. Model systems and experimental conditions that lead to effective repopulation of the liver by transplanted cells. International Journal of Biochemistry and Cell Biology 43:198-213.
22. Lu, L., Li, Y., Kim, S. M., Bossuyt, W., Liu, P., Qiu, Q., Wang, Y., Halder, G., Finegold, M. J., Lee, J. S., et al. 2010. Hippo signaling is a potent in vivo growth and tumor suppressor pathway in the mammalian liver. Proceedings of the National Academy of Sciences of the United States of America 107:1437-1442.
23. Song, H., Mak, K. K., Topol, L., Yun, K., Hu, J., Garrett, L., Chen, Y., Park, O., Chang, J., Simpson, R. M., et al. 2010. Mammalian Mstl and Mst2 kinases play essential roles in organ size control and tumor suppression. Proceedings of the National Academy of Sciences of the United States of America 107:1431-1436.
24. Bai, H., Gayyed, M. F., Lam-Himlin, D. M., Klein, A. P., Nayar, S. K., Xu, Y., Khan, M., Argani, P., Pan, D., and Anders, R. A. 2012. Expression of Yes-associated protein modulates Survivin expression in primary liver malignancies. Human Pathology 43:1376-1385.
25. Feil, R., Wagner, J., Metzger, D., and Chambon, P. 1997. Regulation of Cre recombinase activity by mutated estrogen receptor ligand-binding domains. Biochem Biophys Res Commun 237:752-757.
26. Nguyen, T. H., Oberholzer, J., Birraux, J., Majno, P., Morel, P., and Trono, D. 2002. Highly efficient lentiviral vector-mediated transduction of nondividing, fully reimplantable primary hepatocytes. Mol Ther 6:199-209.
27. Urnov, F. D., Rebar, E. J., Holmes, M. C., Zhang, H. S., and Gregory, P. D. 2010. Genome editing with engineered zinc finger nucleases. Nat Rev Genet 11:636-646.
28. Wright, D. A., Li, T., Yang, B., and Spalding, M. H. 2014. TALEN-mediated genome editing: prospects and perspectives. Biochem J 462:15-24.
29. Mali, P., Yang, L., Esvelt, K. M., Aach, J., Guell, M., DiCarlo, J. E., Norville, J. E., and Church, G. M. 2013. RNA-guided human genome engineering via Cas9. Science 339:823-826.
30. Sakuma, T., and Woltjen, K. 2014. Nuclease-mediated genome editing: At the front-line of functional genomics technology. Dev Growth Differ 56:2-13.
31. Okita, K., Yamakawa, T., Matsumura, Y., Sato, Y., Amano, N., Watanabe, A., Goshima, N., and Yamanaka, S. 2013. An efficient nonviral method to generate integration-free human-induced pluripotent stem cells from cord blood and peripheral blood cells. Stem Cells 31:458-466.
32. Yoshioka, N., Gros, E., Li, H. R., Kumar, S., Deacon, D. C., Maron, C., Muotri, A. R., Chi, N. C., Fu, X. D., Yu, B. D., et al. 2013. Efficient generation of human iPSCs by a synthetic self-replicative RNA. Cell Stem Cell 13:246-254.
33. Nakanishi, M., and Otsu, M. 2012. Development of Sendai virus vectors and their potential applications in gene therapy and regenerative medicine. Curr Gene Ther 12:410-416.
34. Fox I J, D. G., Goldman S A, Huard J, Kamp T J, Trucco M. 2014. Use of differentiated pluripotent stem cells in replacement therapy for treating disease. Science 345:1247391.
35. Xu, R., Kume, A., Matsuda, K. M., Ueda, Y., Kodaira, H., Ogasawara, Y., Urabe, M., Kato, I., Hasegawa, M., and Ozawa, K. 1999. A selective amplifier gene for tamoxifen-inducible expansion of hematopoietic cells. J Gene Med 1:236-244.
36. Block, G. D., Locker, J., Bowen, W. C., Petersen, B. E., Katyal, S., Strom, S. C., Riley, T., Howard, T. A., and Michalopoulos, G. K. 1996. Population expansion, clonal growth, and specific differentiation patterns in primary cultures of hepatocytes induced by HGF/SF, EGF and TGF alpha in a chemically defined (HGM) medium. J Cell Biol 132:1133-1149.
37. Naldini, L., Blomer, U., Gallay, P., Ory, D., Mulligan, R., Gage, F. H., Verma, I. M., and Trono, D. 1996. In vivo gene delivery and stable transduction of nondividing cells by a lentiviral vector. Science 272:263-267.
38. Costa, R. H., Lai, E., Grayson, D. R., and Darnell, J. E., Jr. 1988. The cell-specific enhancer of the mouse transthyretin (prealbumin) gene binds a common factor at one site and a liver-specific factor(s) at two other sites. Mol Cell Biol 8:81-90.
39. Gossrau, R. 1979. [Peptidases II. Localization of dipeptidylpeptidase IV (DPP IV). Histochemical and biochemical study]. Histochemistry 60:231-248.
40. Thompson, N. L., Hixson, D. C., Callanan, H., Panzica, M., Flanagan, D., Faris, R. A., Hong, W. J., Hartel-Schenk, S., and Doyle, D. 1991. A Fischer rat substrain deficient in dipeptidyl peptidase IV activity makes normal steady-state RNA levels and an altered protein. Use as a liver-cell transplantation model. Biochem J 273 (Pt 3):497-502.
41. Fisher, R. A., and Strom, S. C. 2006. Human hepatocyte transplantation: worldwide results. Transplantation 82:441-449.
42. Fitzpatrick, E., Mitry, R. R., and Dhawan, A. 2009. Human hepatocyte transplantation: state of the art. J Intern Med 266:339-357.
43. Fox, I. J., Chowdhury, J. R., Kaufman, S. S., Goertzen, T. C., Chowdhury, N. R., Warkentin, P. I., Dorko, K., Sauter, B. V., and Strom, S. C. 1998. Treatment of the Crigler-Najjar syndrome type I with hepatocyte transplantation. N Engl J Med 338:1422-1426.
44. Ambrosino, G., Varotto, S., Strom, S. C., Guariso, G., Franchin, E., Miotto, D., Caenazzo, L., Basso, S., Carraro, P., Valente, M. L., et al. 2005. Isolated hepatocyte transplantation for Crigler-Najjar syndrome type 1. Cell Transplant 14:151-157.
45. Grossman, M., Rader, D. J., Muller, D. W., Kolansky, D. M., Kozarsky, K., Clark, B. J., 3rd, Stein, E. A., Lupien, P. J., Brewer, H. B., Jr., Raper, S. E., et al. 1995. A pilot study of ex vivo gene therapy for homozygous familial hypercholesterolaemia. Nat Med 1:1148-1154.
46. Guha, C., Parashar, B., Deb, N. J., Garg, M., Gorla, G. R., Singh, A., Roy-Chowdhury, N., Vikram, B., and Roy-Chowdhury, J. 2002. Normal hepatocytes correct serum bilirubin after repopulation of Gunn rat liver subjected to irradiation/partial resection. Hepatology 36:354-362.
47. Nguyen, T. H., Birraux, J., Wildhaber, B., Myara, A., Trivin, F., Le Coultre, C., Trono, D., and Chardot, C. 2006. Ex vivo lentivirus transduction and immediate transplantation of uncultured hepatocytes for treating hyperbilirubinemic Gunn rat. Transplantation 82:794-803.
48. Yovchev, M. I., Xue, Y., Shafritz, D. A., Locker, J., and Oertel, M. 2014. Repopulation of the fibrotic/cirrhotic rat liver by transplanted hepatic stem/progenitor cells and mature hepatocytes. Hepatology 59:284-295.
49. Kume et al. 2003. In vivo expansion of transduced murine hematopoietic cells with a selective amplifier gene J Gene Med 5: 175-181.
50. Nagashima et al. 2004. In vivo expansion of gene-modified hematopoietic cells by a novel selective amplifier gene utilizing the erythropoietin receptor as a molecular switch. J Gene Med 6: 22-31.

| (Nucleotide sequence encoding YAP-ERT2) | |
|---|---|
| SEQ ID NO: 1 | |
| 1 | ATGGACTACAAAGACGATGACGACAAGCTTGCGGCCGCGAATTCAAGCTTAGCCACCATG |
| 61 | GACTACAAAGACGATGACGATAAAGCAAGGCTCGAATCGGTACCTAAGGATCCCGGGCAG |
| 121 | CAGCCGCCGCCTCAACCGGCCCCCCAGGGCCAAGGGCAGCCGCCTTCGCAGCCCCCGCAG |
| 181 | GGGCAGGGCCCGCCGTCCGGACCCGGGCAACCGGCACCCGCGGCGACCCAGGCGGCGCCG |
| 241 | CAGGCACCCCCCGCCGGGCATCAGATCGTGCACGTCCGCGGGGACTCGGAGACCGACCTG |
| 301 | GAGGCGCTCTTCAACGCCGTCATGAACCCCAAGACGGCCAACGTGCCCCAGACCGTGCCC |
| 361 | ATGAGGCTCCGGAAGCTGCCCGACTCCTTCTTCAAGCCGCCGGAGCCCAAATCCCACTCC |
| 421 | CGACAGGCCAGTACTGATGCAGGCACTGCAGGAGCCCTGACTCCACAGCATGTTCGAGCT |
| 481 | CATTCCTCTCCAGCTTCTCTGCAGTTGGGAGCTGTTTCTCCTGGGACACTGACCCCCACT |
| 541 | GGAGTAGTCTCTGGCCCAGCAGCTACACCCACAGCTCAGCATCTTCGACAGTCTTCTTTT |
| 601 | GAGATACCTGATGATGTACCTCTGCCAGCAGGTTGGGAGATGGCAAAGACATCTTCTGGT |
| 661 | CAGAGATACTTCTTAAATCACATCGATCAGACAACAACATGGCAGGACCCCAGGAAGGCC |
| 721 | ATGCTGTCCCAGATGAACGTCACAGCCCCCACCAGTCCACCAGTGCAGCAGAATATGATG |
| 781 | AACTCGGCTTCAGGTCCTCTTCCTGATGGATGGGAACAAGCCATGACTCAGGATGGAGAA |
| 841 | ATTTACTATATAAACCATAAGAACAAGACCACCTCTTGGCTAGACCCAAGGCTTGACCCT |
| 901 | CGTTTTGCCATGAACCAGAGAATCAGTCAGAGTGCTCCAGTGAAACAGCCACCACCCCTG |
| 961 | GCTCCCCAGAGCCCACAGGGAGGCGTCATGGGTGGCAGCAACTCCAACCAGCAGCAACAG |
| 1021 | ATGCGACTGCAGCAACTGCAGATGGAGAAGGAGAGGCTGCGGCTGAAACAGCAAGAACTG |
| 1081 | CTTCGGCAGGAGTTAGCCCTGCGTAGCCAGTTACCAACACTGGAGCAGGATGGTGGGACT |
| 1141 | CAAAATCCAGTGTCTTCTCCCGGGATGTCTCAGGAATTGAGAACAATGACGACCAATAGC |
| 1201 | TCAGATCCTTTCCTTAACAGTGGCACCTATCACTCTCGAGATGAGAGTACAGACAGTGGA |
| 1261 | CTAAGCATGAGCAGCTACAGTGTCCCTCGAACCCCAGATGACTTCCTGAACAGTGTGGAT |
| 1321 | GAGATGGATACAGGTGATACTATCAACCAAAGCACCCTGCCCTCACAGCAGAACCGTTTC |
| 1381 | CCAGACTACCTTGAAGCCATTCCTGGGACAAATGTGGACCTTGGAACACTGGAAGGAGAT |
| 1441 | GGAATGAACATAGAAGGAGAGGAGCTGATGCCAAGTCTGCAGGAAGCTTTGAGTTCTGAC |
| 1501 | ATCCTTAATGACATGGAGTCTGTTTTGGCTGCCACCAAGCTAGATAAAGAAAGCTTTCTT |
| 1561 | ACATGGTTAGTCGACTCTGCTGGAGACATGAGAGCTGCCAACCTTTGGCCAAGCCCGCTC |
| 1621 | ATGATCAAACGCTCTAAGAAGAACAGCCTGGCCTTGTCCCTGACGGCCGACCAGATGGTC |
| 1681 | AGTGCCTTGTTGGATGCTGAGCCCCCCATACTCTATTCCGAGTATGATCCTACCAGACCC |
| 1741 | TTCAGTGAAGCTTCGATGATGGGCTTACTGACCAACCTGGCAGACAGGGAGCTGGTTCAC |
| 1801 | ATGATCAACTGGGCGAAGAGGGTGCCAGGCTTTGTGGATTTGACCCTCCATGATCAGGTC |
| 1861 | CACCTTCTAGAATGTGCCTGGCTAGAGATCCTGATGATTGGTCTCGTCTGGCGCTCCATG |
| 1921 | GAGCACCCAGTGAAGCTACTGTTTGCTCCTAACTTGCTCTTGGACAGGAACCAGGGAAAA |
| 1981 | TGTGTAGAGGGCATGGTGGAGATCTTCGACATGCTGCTGGCTACATCATCTCGGTTCCGC |
| 2041 | ATGATGAATCTGCAGGGAGAGGAGTTTGTGTGCCTCAAATCTATTATTTTGCTTAATTCT |
| 2101 | GGAGTGTACACATTTCTGTCCAGCACCCTGAAGTCTCTGGAAGAGAAGGACCATATCCAC |
| 2161 | CGAGTCCTGGACAAGATCACAGACACTTTGATCCACCTGATGGCCAAGGCAGGCCTGACC |
| 2221 | CTGCAGCAGCAGCACCAGCGGCTGGCCCAGCTCCTCCTCATCCTCTCCCACATCAGGCAC |
| 2281 | ATGAGTAACAAAGGCATGGAGCATCTGTACAGCATGAAGTGCAAGAACGTGGTGCCCCTC |
| 2341 | TATGACCTGCTGCTGGAGGCGGCGGACGCCCACCGCCTACATGCGCCCACTAGCCGTGGA |
| 2401 | GGGGCATCCGTGGAGGAGACGGACCAAAGCCACTTGGCCACTGCGGGCTCTACTTCATCG |
| 2461 | CATTCCTTGCAAAAGTATTACATCACGGGGGAGGCAGAGGGTTTCCCTGCCACAGCTTGA |
| (Amino acid sequence of YAP-ERT2) | |
| SEQ ID NO: 2 | |
| 1 | M D Y K D D D D K L A A A N S S L A T M |
| 21 | D Y K D D D D K A R L E S V P K D P G Q |
| 41 | Q P P P Q P A P Q G Q G Q P P S Q P P Q |
| 61 | G Q G P P S G P G Q P A P A A T Q A A P |
| 81 | Q A P P A G H Q I V H V R G D S E T D L |
| 101 | E A L F N A V M N P K T A N V P Q T V P |
| 121 | M R L R K L P D S F F K P P E P K S H S |
| 141 | R Q A S T D A G T A G A L T P Q H V R A |
| 161 | H S S P A S L Q L G A V S P G T L T P T |
| 181 | G V V S G P A A T P T A Q H L R Q S S F |
| 201 | E I P D D V P L P A G W E M A K T S S G |
| 221 | Q R Y F L N H I D Q T T T W Q D P R K A |
| 241 | M L S Q M N V T A P T S P P V Q Q N M M |
| 261 | N S A S G P L P D G W E Q A M T Q D G E |
| 281 | I Y Y I N H K N K T T S W L D P R L D P |
| 301 | R F A M N Q R I S Q S A P V K Q P P P L |
| 321 | A P Q S P Q G G V M G G S N S N Q Q Q Q |
| 341 | M R L Q Q L Q M E K E R L R L K Q Q E L |
| 361 | L R Q E L A L R S Q L P T L E Q D G G T |
| 381 | Q N P V S S P G M S Q E L R T M T T N S |
| 401 | S D P F L N S G T Y H S R D E S T D S G |
| 421 | L S M S S Y S V P R T P D D F L N S V D |
| 441 | E M D T G D T I N Q S T L P S Q Q N R F |
| 461 | P D Y L E A I P G T N V D L G T L E G D |
| 481 | G M N I E G E E L M P S L Q E A L S S D |
| 501 | I L N D M E S V L A A T K L D K E S F L |
| 521 | T W L V D S A G D M R A A N L W P S P L |
| 541 | M I K R S K K N S L A L S L T A D Q M V |
| 561 | S A L L D A E P P I L Y S E Y D P T R P |
| 581 | F S E A S M M G L L T N L A D R E L V H |
| 601 | M I N W A K R V P G F V D L T L H D Q V |
| 621 | H L L E C A W L E I L M I G L V W R S M |
| 641 | E H P V K L L F A P N L L L D R N Q G K |
| 661 | C V E G M V E I F D M L L A T S S R F R |
| 681 | M M N L Q G E E F V C L K S I I L L N S |
| 701 | G V Y T F L S S T L K S L E E K D H I H |
| 721 | R V L D K I T D T L I H L M A K A G L T |
| 741 | L Q Q Q H Q R L A Q L L L I L S H I R H |
| 761 | M S N K G M E H L Y S M K C K N V V P L |
| 781 | Y D L L L E A A D A H R L H A P T S R G |
| 801 | G A S V E E T D Q S H L A T A G S T S S |
| 821 | H S L Q K Y Y I T G E A E G F P A T A * |
| (Nucleotide sequence encoding YAP-ERT2 plus TTR promoter region) | |
| SEQ ID NO: 3 | |
| 1 | CGCGAGTTAATAATTACCAGCGCGGGCCAAATAAATAATCCGCGAGGGGCAGGTGACGTT |
| 61 | TGCCCAGCGCGCGCTGGTAATTATTAACCTCGCGAATATTGATTCGAGGCCGCGATTGCC |
| 121 | GCAATCGCGAGGGGCAGGTGACCTTTGCCCAGCGCGCGTTCGCCCCGCCCCGGACGGTAT |
| 181 | CGATAAGCTTAGGAGCTTGGGCTGCAGGTCGAGGGCACTGGGAGGATGTTGAGTAAGATG |
| 241 | GAAAACTACTGATGACCCTTGCAGAGACAGAGTATTAGGACATGTTTGAACAGGGGCCGG |
| 301 | GCGATCAGCAGGTAGCTCTAGAGGATCCCCGTCTGTCTGCACATTTCGTAGAGCGAGTGT |
| 361 | TCCGATACTCTAATCTCCCTAGGCAAGGTTCATATTTGTGTAGGTTACTTATTCTCCTTT |
| 421 | TGTTGACTAAGTCAATAATCAGAATCAGCAGGTTTGGAGTCAGCTTGGCAGGGATCAGCA |
| 481 | GCCTGGGTTGGAAGGAGGGGGTATAAAAGCCCCTTCACCAGGAGAAGCCGTCCCGGGAAT |
| 541 | TCAAGCTTAGCCACCATGGACTACAAAGACGATGACGACAAGCTTGCGGCCGCGAATTCA |
| 601 | AGCTTAGCCACCATGGACTACAAAGACGATGACGATAAAGCAAGGCTCGAATCGGTACCT |
| 661 | AAGGATCCCGGGCAGCAGCCGCCGCCTCAACCGGCCCCCCAGGGCCAAGGGCAGCCGCCT |
| 721 | TCGCAGCCCCCGCAGGGGCAGGGCCCGCCGTCCGGACCCGGGCAACCGGCACCCGCGGCG |
| 781 | ACCCAGGCGGCGCCGCAGGCACCCCCCGCCGGGCATCAGATCGTGCACGTCCGCGGGGAC |
| 841 | TCGGAGACCGACCTGGAGGCGCTCTTCAACGCCGTCATGAACCCCAAGACGGCCAACGTG |
| 901 | CCCCAGACCGTGCCCATGAGGCTCCGGAAGCTGCCCGACTCCTTCTTCAAGCCGCCGGAG |
| 961 | CCCAAATCCCACTCCCGACAGGCCAGTACTGATGCAGGCACTGCAGGAGCCCTGACTCCA |
| 1021 | CAGCATGTTCGAGCTCATTCCTCTCCAGCTTCTCTGCAGTTGGGAGCTGTTTCTCCTGGG |
| 1081 | ACACTGACCCCCACTGGAGTAGTCTCTGGCCCAGCAGCTACACCCACAGCTCAGCATCTT |
| 1141 | CGACAGTCTTCTTTTGAGATACCTGATGATGTACCTCTGCCAGCAGGTTGGGAGATGGCA |
| 1201 | AAGACATCTTCTGGTCAGAGATACTTCTTAAATCACATCGATCAGACAACAACATGGCAG |
| 1261 | GACCCCAGGAAGGCCATGCTGTCCCAGATGAACGTCACAGCCCCCACCAGTCCACCAGTG |
| 1321 | CAGCAGAATATGATGAACTCGGCTTCAGGTCCTCTTCCTGATGGATGGGAACAAGCCATG |
| 1381 | ACTCAGGATGGAGAAATTTACTATATAAACCATAAGAACAAGACCACCTCTTGGCTAGAC |
| 1441 | CCAAGGCTTGACCCTCGTTTTGCCATGAACCAGAGAATCAGTCAGAGTGCTCCAGTGAAA |
| 1501 | CAGCCACCACCCCTGGCTCCCCAGAGCCCACAGGGAGGCGTCATGGGTGGCAGCAACTCC |
| 1561 | AACCAGCAGCAACAGATGCGACTGCAGCAACTGCAGATGGAGAAGGAGAGGCTGCGGCTG |
| 1621 | AAACAGCAAGAACTGCTTCGGCAGGAGTTAGCCCTGCGTAGCCAGTTACCAACACTGGAG |
| 1681 | CAGGATGGTGGGACTCAAAATCCAGTGTCTTCTCCCGGGATGTCTCAGGAATTGAGAACA |
| 1741 | ATGACGACCAATAGCTCAGATCCTTTCCTTAACAGTGGCACCTATCACTCTCGAGATGAG |
| 1801 | AGTACAGACAGTGGACTAAGCATGAGCAGCTACAGTGTCCCTCGAACCCCAGATGACTTC |
| 1861 | CTGAACAGTGTGGATGAGATGGATACAGGTGATACTATCAACCAAAGCACCCTGCCCTCA |
| 1921 | CAGCAGAACCGTTTCCCAGACTACCTTGAAGCCATTCCTGGGACAAATGTGGACCTTGGA |
| 1981 | ACACTGGAAGGAGATGGAATGAACATAGAAGGAGAGGAGCTGATGCCAAGTCTGCAGGAA |
| 2041 | GCTTTGAGTTCTGACATCCTTAATGACATGGAGTCTGTTTTGGCTGCCACCAAGCTAGAT |
| 2101 | AAAGAAAGCTTTCTTACATGGTTAGTCGACTCTGCTGGAGACATGAGAGCTGCCAACCTT |
| 2161 | TGGCCAAGCCCGCTCATGATCAAACGCTCTAAGAAGAACAGCCTGGCCTTGTCCCTGACG |
| 2221 | GCCGACCAGATGGTCAGTGCCTTGTTGGATGCTGAGCCCCCCATACTCTATTCCGAGTAT |
| 2281 | GATCCTACCAGACCCTTCAGTGAAGCTTCGATGATGGGCTTACTGACCAACCTGGCAGAC |
| 2341 | AGGGAGCTGGTTCACATGATCAACTGGGCGAAGAGGGTGCCAGGCTTTGTGGATTTGACC |
| 2401 | CTCCATGATCAGGTCCACCTTCTAGAATGTGCCTGGCTAGAGATCCTGATGATTGGTCTC |
| 2461 | GTCTGGCGCTCCATGGAGCACCCAGTGAAGCTACTGTTTGCTCCTAACTTGCTCTTGGAC |
| 2521 | AGGAACCAGGGAAAATGTGTAGAGGGCATGGTGGAGATCTTCGACATGCTGCTGGCTACA |
| 2581 | TCATCTCGGTTCCGCATGATGAATCTGCAGGGAGAGGAGTTTGTGTGCCTCAAATCTATT |
| 2641 | ATTTTGCTTAATTCTGGAGTGTACACATTTCTGTCCAGCACCCTGAAGTCTCTGGAAGAG |
| 2701 | AAGGACCATATCCACCGAGTCCTGGACAAGATCACAGACACTTTGATCCACCTGATGGCC |
| 2761 | AAGGCAGGCCTGACCCTGCAGCAGCAGCACCAGCGGCTGGCCCAGCTCCTCCTCATCCTC |
| 2821 | TCCCACATCAGGCACATGAGTAACAAAGGCATGGAGCATCTGTACAGCATGAAGTGCAAG |
| 2881 | AACGTGGTGCCCCTCTATGACCTGCTGCTGGAGGCGGCGGACGCCCACCGCCTACATGCG |
| 2941 | CCCACTAGCCGTGGAGGGGCATCCGTGGAGGAGACGGACCAAAGCCACTTGGCCACTGCG |
| 3001 | GGCTCTACTTCATCGCATTCCTTGCAAAAGTATTACATCACGGGGGAGGCAGAGGGTTTC |
| 3061 | CCTGCCACAGCTTGA |

1. A method for repopulating the liver in a patient having a liver disease or condition, comprising:
(a) administering a plurality of modified normal liver cells obtained from a deceased or living donor, wherein the modified cells comprise a nucleic acid molecule encoding a Yes-associated protein-estrogen receptor 2 (YAP-ERT2) fusion protein; and
(b) administering an estrogen receptor antagonist, wherein the estrogen receptor antagonist increases the proliferative activity of the modified cells, thereby repopulating the liver with the modified cells.

2. The method of claim 1, wherein administration of the estrogen receptor antagonist induces nuclear translocation of the YAP-ERT2 fusion protein where it functions as a transcriptional coactivator of Yap target genes.

3. (canceled)

4. The method of claim 1, wherein administration of the modified cells comprises transplantation of the cells into the liver of the patient.

5-6. (canceled)

7. The method of claim 1, wherein the modified cells repopulate the liver cell population in the patient by about 8-12% or greater at about six months following administration of the modified cells.

8-10. (canceled)

11. The method of claim 1, wherein the estrogen receptor antagonist is tamoxifen and is administered at a dosage of about 20 mg/day to about 40 mg/day.

12-15. (canceled)

16. The method of claim 1, wherein the estrogen receptor antagonist is administered for 1, 2, 3, 4, 5, 6, 7, 8, 9, 10, 11, 12 months or longer.

17-21. (canceled)

22. The method of claim 1, wherein the nucleic acid molecule encoding the YAP-ERT2 fusion protein comprises a nucleotide sequence set forth in SEQ ID NO: 1.

23. The method of claim 1, wherein the YAP-ERT2 fusion protein comprises a polypeptide having the amino acid sequence set forth in SEQ ID NO: 2.

24. The method of claim 1, wherein the nucleic acid molecule encoding the YAP-ERT2 fusion protein is operably linked to a promoter.

25-26. (canceled)

27. The method of claim 24, wherein the promoter is a liver specific promoter.

28. (canceled)

29. The method of claim 1, wherein the cells are primary hepatocytes, an hepatic cell line, stem cells from the liver or other sources, such as embryonic stem (ES) cells or induced pluripotent stem (iPS) cells, that have been transduced with a lentivirus, a retrovirus, an adeno-associated virus or a Sendai virus containing the YapERT2 sequence or by introducing the YapERT2 sequence into the cells using a zinc finger nuclease (ZFN), a transcription activator-like effector nuclease (TALEN), or clustered regulatory interspaced short palindromic repeat (CRISPR)/Cas-based RNA-guided DNA endonuclease technology, a recombinant Epstein Barr Nuclear Antigen plasmid, a self-replicating RNA molecule or other method that allows uptake and function of nucleic acids in cells.

30-35. (canceled)

36. The method of claim 1, wherein the liver disease or condition is a genetic based disease.

37. The method of claim 36, wherein the liver disease or condition is selected from among UDP-glucuronosyl transferase deficiency (Crigler-Najjar Syndrome, Type 1), ornithine transcarbamylase deficiency, Familial Hypercholesterolemia, phenylketonuria, Hemophilia B, Factor VII deficiency, primary hyperoxaluria, maple syrup urine disease, Apolipoprotein E deficiency, Wilson's Disease, al-antitrypsin deficiency, Hereditary Hemochromatosis, Progressive Familial Intrahepatic Cholestasis (Types I, II and III), and Bile Salt Export Protein deficiency.

38-40. (canceled)

41. The method of claim 1, wherein the liver disease or condition is selected from among a non-genetic based liver disease, such as but not limited to non-alcoholic fatty liver disease and chronic hepatitis C virus infection.

42. The method of claim 41, wherein the patient exhibits hepatic fibrosis or cirrhosis.

43-45. (canceled)

46. The method of claim 1, further comprising administration of an immunosuppressant.

47. The method of claim 1, further comprising administration of an additional therapeutic agent.

48. The method of claim 47, wherein the therapeutic agent is an anti-fibrotic agent, selected from among Sorafemib, largazole, galectin inhibitors, FG-3019 (an anti-CTGF antibody), Pirfenidone, a TGF-inhibitor, endostatin peptide, and Polarezin.

49-52. (canceled)

53. The method of claim 1, wherein the modified cells further express a therapeutic gene.

54-56. (canceled)

57. A method for repopulating the liver in a patient having a genetic-based liver disease or condition, comprising:
(a) administering a plurality of modified cells that have been isolated from the patient wherein the modified cells comprise: (i) a nucleic acid molecule encoding a Yes-associated protein-estrogen receptor 2 (YAP-ERT2) fusion protein and (ii) a nucleic acid molecule encoding a protein that is deficient or defective in the patient; and
(b) administering an estrogen receptor antagonist, wherein the estrogen receptor antagonist increases the proliferative activity of the modified cells, thereby repopulating the liver with the modified cells while avoiding immunosuppressive therapy.

58-99. (canceled)

100. A method of repopulating the cells of a tissue or organ comprising:
introducing a plurality of modified cells into a tissue or organ sufficient to effect about 3-10% or greater repopulation of the cells in the tissue or organ under non-selective conditions within about 3-6 months, wherein the modified cells are modified to increase their proliferative potential compared to unmodified cells in the presence of a ligand; and
optionally, administering the ligand to the patient into which the modified cells have been transplanted.

101-112. (canceled)

113. A method for treating diabetes in a patient, comprising:
(a) administering to the patient a plurality of modified pancreatic islets, pancreatic islet β cells, stem cells, such as embryonic stem (ES) cells or induced pluripotent stem (iPS) cells, which have been transduced with a lentivirus, a retrovirus, an adeno-associated virus or a Sendai virus containing the YapERT2 sequence or by introducing the YapERT2 sequence into the cells using a zinc finger nuclease (ZFN), a transcription activator-like effector nuclease (TALEN), or clustered regulatory interspaced short palindromic repeat (CRISPR)/Cas-based RNA-guided DNA endonuclease technology, a recombinant Epstein Barr Nuclear Antigen plasmid, a self-replicating RNA molecule or other method that allows uptake and function of nucleic acids in cells, and which have been differentiated along the pancreatic islet 0 cell lineage pathway; and
(b) administering an estrogen receptor antagonist that activates Yap ERT2 fusion protein function and induces cell proliferation, thereby treating the diabetes.

114. The method of claim 113, wherein the nucleic acid encoding the YAP-ERT2 fusion protein is operably linked to a pancreatic islet β cell-specific promoter.

115-131. (canceled)
